(19) United States
(12) Patent Application Publication
(10) Pub. No.: US 2024/0026362 A1
(43) Pub. Date: Jan. 25, 2024

(54) INTERFERING RNAS TARGETING SEVERE ACUTE RESPIRATORY SYNDROME-ASSOCIATED CORONAVIRUS AND USES THEREOF FOR TREATING COVID-19

(71) Applicants: MICROBIO (SHANGHAI) CO. LTD., Shanghai (CN); ONENESS BIOTECH CO. LTD., Taipei (TW)

(72) Inventors: Yi-Chung CHANG, Taipei (TW); Chi-Fan YANG, Shanghai (CN); Yi-Fen CHEN, Shanghai (CN); Chia-Chun YANG, Taipei (TW); Yuan-Lin CHOU, Shanghai (CN)

(73) Assignees: MICROBIO (SHANGHAI) CO. LTD., Shanghai (CN); ONENESS BIOTECH CO. LTD., Taipei (TW)

(21) Appl. No.: 18/255,690

(22) PCT Filed: Dec. 3, 2021
(86) PCT No.: PCT/CN2021/135476

§ 371 Date: Jun. 2, 2023

(30) Foreign Application Priority Data
Dec. 3, 2020 (WO) PCT/CN2020/133565
Sep. 29, 2021 (WO) PCT/CN2021/121762

(51) Int. Cl. C12N 15/113; A61K 39/215; A61K 39/42; A61K 31/675; A61P 31/14

(52) U.S. Cl. CPC C12N 15/1131; A61K 39/215; A61K 39/42; A61K 31/675; A61P 31/14; C12N 2310/14

(57) ABSTRACT

Provided are interfering RNAs (e.g., siRNAs) targeting SARS-CoV (e.g., the POL, Spike, Helicase, or Envelop gene thereof) and therapeutic uses thereof for inhibiting SARS-CoV infection and/or treating diseases associated with the infection (e.g., COVID-19).

### CROSS REFERENCE TO RELATED APPLICATIONS

This application claims the benefit of the filing dates of International Application No. PCT/CN2020/133565, filed Dec. 3, 2020 and International Application No. PCT/CN2021/121762, filed Sep. 29, 2021, the entire contents of each of which are incorporated by reference herein.

### BACKGROUND OF THE INVENTION

Coronaviruses, members of the family Coronaviridae and subfamily Coronavirinae, are enveloped viruses containing single-strand, positive-sense RNA genome ranging from 26 to 32 kilobases in length. Coronaviruses have been identified in several vertebrate hosts including bird, bat, pig, rodent, camel, and human. Human can acquire coronavirus infection from other host of mammals, which may cause detrimental upper respiratory tract illness.

Members of the coronaviruses family include virus strains having different phylogenetic origin and causing different severity in mortality and morbidity. As such, treatment for coronavirus infection varies depending on the specific strains, for example, SARS-CoV-2 variants (e.g., the Delta variant) that cause the infection. So far, there is no approved antiviral drug treatment for coronavirus infection. Accordingly, there is a need for developing treatment of coronavirus infection, particularly treatment of infection caused by severe acute respiratory syndrome (SARS)-associated coronavirus (SARS-CoV), e.g., treatment of COVID-19.

### SUMMARY OF THE INVENTION

The present disclosure is based, at least in part, on the development of interfering RNA molecules targeting the genomic RNA of a SARS-CoV virus, for example, SARS-CoV-1 or SARS-CoV-2. The interfering RNA molecules disclosed herein exhibited high efficiency in inhibiting SARS-CoV-2 replication and production. Accordingly, provided herein are anti-SARS-CoV-2 interfering RNAs (e.g., siRNAs) and uses thereof for inhibiting a SARS-CoV-2 virus and for treating COVID-19.

In some aspects, the present disclosure provides a method for inhibiting a SARS-CoV virus (e.g., SARS-CoV-1 or SARS-CoV-2), the method comprising: contacting an effective amount of a small interfering RNA (siRNA) with a cell infected with a SARS-CoV virus, wherein the siRNA targets a genomic site of the SARS-CoV virus. In some embodiments, the siRNA may target a SARS-CoV (e.g., SARS-CoV-1 or SARS-CoV-2) POL gene, Spike gene, Helicase gene, or Envelop gene. In some embodiments, the siRNA may target a SARS-CoV genomic site (e.g., a genomic site of SARS-CoV-1 or SARS-CoV-2) comprising one of the following nucleotide sequences (e.g., (vi)-(viii), (x), or (xi)):

(i)
(SEQ ID NO: 2)
5′-GAGGCACGUCAACAUCUUA-3′,
(ii)
(SEQ ID NO: 4)
5′-CAGCAUUAAAUCACACUAA-3′,
(iii)
(SEQ ID NO: 6)
5′-CGGUGUUUAAACCGUGUUU-3′,
(iv)
(SEQ ID NO: 8)
5′-GUGGUACAACUACACUUAA-3′,
(v)
(SEQ ID NO: 10)
5′-UGGCUUGAUGACGUAGUUU-3′,
(vi)
(SEQ ID NO: 12)
5′-CUGUCAAACCCGGUAAUUU-3′,
(vii)
(SEQ ID NO: 14)
5′-GCGGUUCACUAUAUGUUAA-3′,
(viii)
(SEQ ID NO: 16)
5′-GCCACUAGUCUCUAGUCAG-3′,
(ix)
(SEQ ID NO: 18)
5′-CUCCUACUUGGCGUGUUUA-3′,
(x)
(SEQ ID NO: 20)
5′-CGCACAUUGCUAACUAAGG-3′,
and
(xi)
(SEQ ID NO: 22)
5′-CAGGUACGUUAAUAGUUAA-3′.

As used herein, an siRNA targeting a genomic site of a SARS-CoV virus (e.g., SARS-CoV-1 or SARS-CoV-2) means that the siRNA comprises a fragment that is complementary to the genomic site (completely or partially) such that the siRNA can interact with the genomic RNA of the virus or a messenger RNA (mRNA) comprising a region transcribed by the genomic site to exert its inhibitory activity, e.g., inhibiting viral genome replication and/or down-regulating expression of the encoded protein product. In some instances, the siRNA targets a site of an mRNA synthesized by the SARS-CoV virus.

In some embodiments, the siRNA disclosed herein may target a site within a RNA-dependent RNA polymerase (RdRP) of the SARS-CoV virus (e.g., target a site with the RdRP messenger RNA (mRNA)). Such a siRNA may target a site in the RdRP mRNA within the nucleotide sequence of 5′-UUGCUUUUCAAACUGUCAAACCCGGUAAUUUUAACAAAGA-3′ (SEQ ID NO: 23). In some examples, the siRNA may target a site within the nucleotide sequence of

(SEQ ID NO: 24)
5′-UUUCAAACUGUCAAACCCGGUAAUUUU-3′.

In some examples, the siRNA is a double-strand molecule comprising a sense chain and an antisense chain. Exemplary sense chains and the antisense chains for exemplary anti-SARS-CoV (e.g., SARS-CoV-1 or SARS-CoV-2) are provided below:

(i)
(SEQ ID NO: 25)
5′-GAGGCACGUCAACAUCUUX₁-3′
and
(SEQ ID NO: 26)
5′-X₂AAGAUGUUGACGUGCCUCN₁N₂-3′;
(ii)
(SEQ ID NO: 27)
5′-CAGCAUUAAAUCACACUAX₁-3′,
and
(SEQ ID NO: 28)
5′-X₂UAGUGUGAUUUAAUGCUGN₁N₂-3′;
(iii)
(SEQ ID NO: 29)
5′-CGGUGUUUAAACCGUGUUX₁-3′,
and
(SEQ ID NO: 30)
5′-X₂AACACGGUUUAAACACCGN₁N₂-3′;
(iv)
(SEQ ID NO: 31)
5′-GUGGUACAACUACACUUAX₁-3′,
and
(SEQ ID NO: 32)
5′-X₂UAAGUGUAGUUGUACCACN₁N₂-3′;
(v)
(SEQ ID NO: 33)
5′-UGGCUUGAUGACGUAGUUX₁-3′,
and
(SEQ ID NO: 34)
5′-X₂AACUACGUCAUCAAGCCAN₁N₂-3′;
(vi)
(SEQ ID NO: 35)
5′-CUGUCAAACCCGGUAAUUX₁-3′,
and
(SEQ ID NO: 36)
5′-X₂AAUUACCGGGUUUGACAGN₁N₂-3′;
(vii)
(SEQ ID NO: 37)
5′-GCGGUUCACUAUAUGUUAX₁-3′,
and
(SEQ ID NO: 38)
5′-X₂UAACAUAUAGUGAACCGCN₁N₂-3′;
(viii)
(SEQ ID NO: 39)
5′-GCCACUAGUCUCUAGUCAX₁-3′,
and
(SEQ ID NO: 40)
5′-X₂UGACUAGAGACUAGUGGCN₁N₂-3′;
(ix)
(SEQ ID NO: 41)
5′-CUCCUACUUGGCGUGUUUX₁-3′,
and
(SEQ ID NO: 42)
5′-X₂AAACACGCCAAGUAGGAGN₁N₂-3′;
(x)
(SEQ ID NO: 43)
5′-CGCACAUUGCUAACUAAGX₁-3′,
and
(SEQ ID NO: 44)
5′-X₂CUUAGUUAGCAAUGUGCGN₁N₂-3′;
or
(xi)
(SEQ ID NO: 45)
5′-CAGGUACGUUAAUAGUUAX₁-3′,
and
(SEQ ID NO: 46)
5′-X₂UAACUAUUAACGUACCUGN₁N₂-3′.

X₁ and X₂ in each of the sense chain and antisense chain of each of (i)-(xi), independently, are A and U, respectively or vice versa. Alternatively, X₁ and X₂ are G and C, respectively, or vice versa. Each of N₁ and N₂ in each of the sense chain and antisense chain of each of (i)-(xi), independently, is A, U, G, or C. In some examples, N₂ can be U.

In some examples, the sense chains and the antisense chains for exemplary anti-SARS-CoV (e.g., anti-SARS-CoV-1 or anti-SARS-CoV-2) are provided below:

(i)
(SEQ ID NO: 25)
5′-GAGGCACGUCAACAUCUUX₁-3′
and
(SEQ ID NO: 47)
5′-X₂AAGAUGUUGACGUGCCUCUU-3′;
(ii)
(SEQ ID NO: 27)
5′-CAGCAUUAAAUCACACUAX₁-3′,
and
(SEQ ID NO: 48)
5′-X₂UAGUGUGAUUUAAUGCUGUU-3′;
(iii)
(SEQ ID NO: 29)
5′-CGGUGUUUAAACCGUGUUX₁-3′,
and
(SEQ ID NO: 49)
5′-X₂AACACGGUUUAAACACCGUU-3′;
(iv)
(SEQ ID NO: 31)
5′-GUGGUACAACUACACUUAX₁-3′,
and
(SEQ ID NO: 50)
5′-X₂UAAGUGUAGUUGUACCACUU-3′;
(v)
(SEQ ID NO: 33)
5′-UGGCUUGAUGACGUAGUUX₁-3′,
and
(SEQ ID NO: 51)
5′-X₂AACUACGUCAUCAAGCCAUU-3′;
(vi)
(SEQ ID NO: 35)
5′-CUGUCAAACCCGGUAAUUX₁-3′,
and
(SEQ ID NO: 52)
5′-X₂AAUUACCGGGUUUGACAGUU-3′;
(vii)
(SEQ ID NO: 37)
5′-GCGGUUCACUAUAUGUUAX₁-3′,
and
(SEQ ID NO: 53)
5′-X₂UAACAUAUAGUGAACCGCUU-3′;
(viii)
(SEQ ID NO: 39)
5′-GCCACUAGUCUCUAGUCAX₁-3′,
and
(SEQ ID NO: 54)
5′-X₂UGACUAGAGACUAGUGGCUU-3′;
(ix)
(SEQ ID NO: 41)
5′-CUCCUACUUGGCGUGUUUX₁-3′,
and
(SEQ ID NO: 55)
5′-X₂AAACACGCCAAGUAGGAGUU-3′;
(x)
(SEQ ID NO: 43)
5′-CGCACAUUGCUAACUAAGX₁-3′,
and
(SEQ ID NO: 56)
5′-X₂CUUAGUUAGCAAUGUGCGUU-3′;
or
(xi)
(SEQ ID NO: 45)
5′-CAGGUACGUUAAUAGUUAX₁-3′,
and
(SEQ ID NO: 57)
5′-X₂UAACUAUUAACGUACCUGUU-3′;

wherein X₁ and X₂ in each of the sense chain and antisense chain of each of (i)-(xi), independently, are A and U, respectively, or vice versa. Specific examples are provided in Table I below.

In some examples, the sense chain and the antisense chain may comprise the nucleotide sequences set forth in (vi), (vii), (viii), (x), or (xi). In specific examples, the siRNA can be C6. In other examples, the siRNA can be C7. In other examples, the siRNA can be C8. In other examples, the siRNA can be C10. In other examples, the siRNA can be C11. As used herein, siRNAs C6, C7, C10, C11, etc. refer to siRNAs comprising the antisense and sense sequences corresponding to each siRNA provided herein, regardless of their modification profiles. For example, siRNA C6 refers to siRNAs having a sense strand comprising SEQ ID NO: 12 and an antisense strand comprising SEQ ID NO: 11, one or both of which can be either unmodified or modified in any pattern (e.g., those disclosed herein).

Any of the interfering RNAs (e.g., siRNAs) disclosed herein may comprise one or more modified nucleotides. In some examples, the one or more modified nucleotides comprise 2′-fluoro, 2′-O-methyl, or a combination thereof. Alternatively or in addition, the interfering RNAs (e.g., siRNAs) disclosed herein may comprise a modified backbone, for example, comprising one or more phosphorothioate bonds. For example, the modified siRNA may be C6G25S. In other examples, the modified siRNA may be C8G25S. In yet other examples, the modified siRNA may be C10G31A.

In any of the methods disclosed herein, the contacting step is performed by administering the siRNA to a subject having infected by the SARS-CoV virus. In some examples, the subject may be infected by SARS-CoV-1. In other examples, the subject may be infected by SARS-CoV-2 (e.g., having COVID19). The siRNA can be formulated in a pharmaceutical composition, which further comprises a pharmaceutically acceptable carrier. In some instances, the subject can be a human patient infected with a SARS-CoV virus, e.g., infected by SARS-CoV-1 or SARS-CoV-2. In other instances, the subject may be a human patient suspected of having SARS-CoV infection. In yet other instances, the subject may be a human patient at risk for such infection. In some instances, the subject may be further administered an agent for treatment of infection caused by the SARS-CoV, e.g., infection caused by SARS-CoV-1 or SARS-CoV-2. Examples include, but are not limited to, an anti-SARS-CoV antibody, an anti-SARS-CoV vaccine (e.g., an mRNA vaccine), remdesivir, a steroid, or a combination thereof.

In some instances, the siRNAs targeting SARS-CoV (e.g., SARS-CoV-1 or SARS-CoV-2) as disclosed herein or a pharmaceutical composition comprising such may be delivered to a subject in need of the treatment via a nasal route, for example, intranasal instillation or aerosol inhalation. In specific examples, the siRNAs targeting SARS-CoV (e.g., SARS-CoV-1 or SARS-CoV-2) as disclosed herein or a pharmaceutical composition comprising such may be delivered to a subject by both intranasal instillation and aerosol inhalation.

Any of the methods disclosed herein may further comprise administering to the subject any of the agents for treatment of infection caused by the SARS-CoV virus as disclosed herein (e.g., SARS-CoV-1 or SARS-CoV-2).

In other aspects, provided herein are any of the siRNAs disclosed herein that targets a SARS-CoV-2 virus, for example, C6, C7, C8, C10, or C11, and pharmaceutical compositions comprising such and a pharmaceutically acceptable carrier. In some embodiments, the siRNAs are modified by, e.g., any pattern disclosed herein. In specific examples, the siRNAs are modified siRNAs of C6G25S, C8G25S, or C10G31A.

Also within the scope of the present disclosure are any of the siRNAs or pharmaceutical compositions comprising such for use in inhibiting SARS-CoV (e.g., SARS-CoV-1 or SARS-CoV-2) infection and/or for treating diseases caused by the infection (e.g., COVID-19), as well as uses of such siRNA or pharmaceutical composition for manufacturing a medicament for use in inhibiting SARS-CoV (e.g., SARS-CoV-1 or SARS-CoV-2) infection and/or for treating diseases caused by the infection, for example, COVID-19.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph illustrating the inhibitory activities of exemplary siRNAs as indicated against SARS-CoV-2 proliferation in Vero cells, as determined by RT-qPCR.

FIG. 2 is a graph illustrating the reduction of virus production, as quantified by plaque assay, in the presence of the exemplary siRNAs as indicated.

FIGS. 3A-3E include diagrams showing the identification of highly potent siRNAs against SARS-CoV-2. FIG. 3A: a flowchart illustrating the selection strategy used for identifying potent siRNAs targeting SARS-CoV-2. The selection criteria and numbers of hits remaining at the end of each stage were indicated. FIG. 3B: a graph showing the viral E gene expression in Vero E6 cells. Vero E6 cells were pre-transfected with 10 nM of siRNA and SARS-CoV-2 was added after 24h at a multiplicity of infection (MOI) of 0.1. The numbers of viral RNA copies were quantified with RT-qPCR. The control is scrambled siRNA and is abbreviated as “Ctrl.” C1-C11 are the final candidate sequences after selection. FIG. 3C: a graph showing the plaque inhibition in Vero E6 cells. Vero E6 cells were pre-transfected with 10 nM of siRNA and SARS-CoV-2 was added after 24 h at a multiplicity of infection (MOI) of 0.1. The number of infectious virions were quantified with plaque-forming assay. FIG. 3D: a graph illustrating IC 50 of C6 and the fully modified C6G25S. Vero E6 cells were transfected with 10, 2, 0.4, 0.08, or 0.016 nM of C6 or C6G25S and challenged with virus at MOI of 0.2. Viral genes were quantified by RT-qPCR at 24 h after infection. FIG. 3E: shows a graph illustrating IC 50 data for viral RdRp inhibition by C6G25S. Vero E6 cells were transfected with 10, 2, 0.4, 0.08, or 0.016 nM of C6G25S and challenged with virus at MOI of Viral genes were quantified by RT-qPCR at 24 h after infection.

FIGS. 4A-4B include diagrams showing that C6G25S targeted and inhibited various strains of SARS-CoV-2. FIG. 4A: a genome map for four VOC, four VOI, and two other variants. It shows that C6 targets a highly conserved region of the virus RdRp (accession number: NC_045512.2). Spots above the genome indicate the locations of typical mutations for each variant. Important mutations in the spike protein associated with either viral infectivity or resistance to the immune system are labeled in red, and mutated amino acids are as indicated. Other mutations are labeled in black. The target site and sequence for C6G25 recognition of RdRP is shown below the map. The sequence of the antisense of C6G25 corresponds to SEQ ID NO: 58 and the viral genome region comprising the target site sequence corresponding to SEQ ID NO: 59. FIG. 4B: is a graph showing IC 50 for C6G25S against different variants. Vero E6 cells were transfected with 10, 2, 0.4, 0.08, or 0.016 nM of C6G25S and challenged with different strains of virus. Viral genes were quantified by RT-qPCR at 24 h after infection.

FIGS. 5A-5F include diagrams showing the in vivo study of administration route for C6G25S. FIG. 5A: a photo showing the distribution of C6G25S in lungs via AI. K18-hACE2-transgenic mice treated with C6G25S by 1.48 mg/L of AI for 30 min. FIG. 5B: a photo showing the distribution of C6G25S in lungs via IN. K18-hACE2-transgenic mice treated with 50 ul of PBS containing 50 ug of C6G25S by IN. FIG. 5C: a photo showing the distribution in lung of mice treated with PBS without C6G25S treatment served as a negative control (n=5 per group). C6G25S distribution in lungs was visualized by ISH staining with C6G25S-specific probe (red color). Bronchi (i) and bronchioles (ii) marked with the boxes are enlarged on the right. Figure a graph illustrating quantification of C6G25S-positive cells in lungs of K18-hACE2-transgenic mice (NC=negative control; IN=intranasal instillation; AI=aerosol inhalation). FIG. 5E: a graph illustrating the siRNA level deposited in lungs and nasal cavities of C57/B6 mice after delivery via AI. FIG. 5F: a graph illustrating the siRNA level deposited in lungs and nasal cavities of C57/B6 mice after delivery via IN. (n=3 per group). Quantification data represent mean±SD.

FIGS. 6A-6C include diagrams showing the quantification of C6G25S in inhalation aerosol, lungs, and nasal cavities. FIG. 6A: a graph illustrating Bₘₐₓ. Bₘₐₓ represents the maximum C6G25S level. After aerosol was generated, aerosol samples were collected from the inhalation chamber using 0.5 mL syringes and passed through 100 uL nuclease-free water. C6G25S level in the nuclease-free water was subsequently determined by OD260. FIG. 6B: a graph illustrating quantification of C6G25S level in lung at 0.5, 8, 24, and 48 hr post-delivery via both aerosol inhalation and intranasal instillation. FIG. 6C: a graph illustrating quantification of C6G25S level in nasal cavity at 0.5, 8, 24, and 48 hr post-delivery via both aerosol inhalation and intranasal instillation. C57/B6 mice (n=3 per group) were administrated with 0.74 mg/L of C6G25S via AI for 30 min and followed by 50 ug C6G25S via IN. Quantification data represent mean±SD.

FIGS. 7A-7D include diagrams showing the prophylatic and post-exposure administration of C6G25S in treatment of SARS-CoV-2 and Delta variant in vivo. FIG. 7A: diagrams showing virus levels in control and treated mice not infected with the virus. Left panel is a graph illustrating the viral RNA in lungs of K18-hACE2-transgenic mice quantified with RT-qPCR and plaque forming assay, respectively, at 2 dpi. Right panel is a graph illustrating the infectious virons in lungs of K18-hACE2-transgenic mice quantified with RT-qPCR and plaque forming assay, respectively, at 2 dpi. P-value by Student t test. K18-hACE2-transgenic mice (Winkler, et al., 2020, Nat Immunol 21: 1327-1335) were treated once daily for 3 days before challenging intranasally with 104 plaque-forming units (PFU) of the original virus. Prophylactic treatment consists of 30 min of AI (1.48 mg/l of C6G25S) and followed by IN of 50 ug C6G25S. FIG. 7B: diagrams showing virus levels in control and treated mice infected with the Delta variant of SARS-CoV-2. Left panel is a graph illustrating the quantification of viral RNA in lungs of K18-hACE2-transgenic mice post-exposure. Right panel is a graph illustrating the quantification of infectious virons in lungs of K18-hACE2-transgenic mice post-exposure. Mice were challenged intranasally with 104 PFU of virus and postexposure treated with 2.96 mg/L of C6G25S by AI for 30 min on day 0 (right after infection) and day 1. Viral RNA and infectious virions were quantified at 2 dpi. FIG. 7C: diagrams showing virus levels in control and treated mice not infected following the same experimental design as in FIG. 7A. Viral RNA (left panel) and infectious virions (right panel) in lungs were quantified at 2 dpi. FIG. 7D: graphs showing postexposure treatment of C6G25S against Delta virus. The two-dose group was treated at day 0, and day 1, and analyzed at day 2 dpi. A three-dose group was treated at day 0, day 1, and day 2, and analyzed at day 3 dpi. Virus RNA level (left panel) and infectious virons (right panel) was assessed relative to controls of each time-point. Treatment group are labeled as T and buffer control labeled as C.

FIGS. 8A-8C include diagrams showing that C6G25S prevents SARS-CoV-2-induced tissue damage in the lungs of K18-hACE2 transgenic mice. FIG. 8A: a graph showing the quantitative analysis of ISH images from lung. Whole lung section per mice and 5 mice per group were measured. Data represent mean±SD, P-value by Student t test. FIG. 8B: a graph showing the quantitative analysis of lung-infiltrated immune cells in whole sections of lung. Percentage of positively stained area for control group (n=5) was measured and normalized to 100. The relative percentage of positively stained area for untreated control was shown in red and C6G25S-treated group shown in blue (n=5). Data represent mean±SD, P-value by Student t test. FIG. 8C: a graph showing the lung injury scores were calculated for 5 mice per groups. Data represent mean±SD, P-value by Student t test.

FIG. 9 is a diagram showing the clinical utility of C6G25S. No stimulation of inflammatory cytokines in peripheral blood mononuclear cells (PBMCs) after cocultured with 10 uM of modified C6 (C6G25S) and C8 (C8G25S) siRNA. Cytokines IL-1 alpha, IL-1 beta, IL-6, IL-10, TNF-alpha, and IFN-gamma in the coculture medium were detected via flow cytometry analysis using Cytometric Bead Assay (CBA) Flex Set (BD Biosciences). CpG and poly(I:C) were utilized as positive controls. Data are presented as the means±SD of two independent experiments using PBMCs from three healthy donors. Conc.=concentration.

FIG. 10 is a graph illustrating the effect of C6G25S on the cell viability of BEAS-2B cells measured by CCK-8 assay. Compared with the untreated group, there was no significant cytotoxicity at up to 40 uM of C6G25S.

FIGS. 11A-11B include diagrams depicting single and repeated-dose toxicology study of C6G25S. FIG. 11A: a graph illustrating single-dose toxicology study. A single dose of C6G25S (0, 20, 40, and 75 mg/kg) was administered intranasally to Sprague Dawley rats at day 0 (n=3 per group). Body weight and food intake were monitored daily for 7 days. FIG. 11B: is a graph illustrating repeated-dose toxicology study. ICR mice (n=3 per group) were administered daily with the indicated concentration of C6G25S by intranasal instillation and continuously monitored body weight and food intake for 14 days.

FIG. 12 is a flowchart illustrating potential mechanism of action of C6G25S.

FIGS. 13A-13B include diagrams depicting that miR2911 with one of the binding sites overlaps with that of C6 reduced viral RNA of original virus, but not alpha variant. FIG. 13A: a diagram showing the locations targeted by all 11 siRNA candidates within the SARS-CoV-2 genome (accession number: NC_045512.2). The overlapping target sites of C6 and miR2911 on RdRp are depicted with the sequences of C6 antisense and miR2911 outlined in red and blue, respectively. Sequences are SEQ ID NOs: 60, 61, and 58, from top to bottom. FIG. 13B: a graph showing the inhibition of viral RNA caused by miR2911. Vero E6 cells were transfected with 100 nM of miR2911 and then infected with original virus and alpha variant at a MOI of 0.1, respectively. Viral RNA was detected by RT-qPCR.

### DETAILED DESCRIPTION OF THE INVENTION

RNA interference or “RNAi” is a process in which double-stranded RNAs (dsRNA) block gene expression when it is introduced into host cells. (Fire et al. (1998) Nature 391, 806-811). Short dsRNA directs gene-specific, post-transcriptional silencing in many organisms, including vertebrates, and has provided a new tool for studying gene function. RNAi may involve mRNA degradation.

The present disclosure is based, at least in part, on the development of interfering RNAs targeting the RNA genome of SARS-CoV virus. Such interfering RNAs may target the RNA genome of SARS-CoV-1 in some instances. In other instances, the interfering RNA may target the RNA genome of SARS-CoV-2. Via RNA interference, such interfering RNAs showed high efficiency in inhibiting SARS-CoV RNA genome replication and virus production in Vero cells, indicating their therapeutic potentials in inhibiting SARS-CoV infection and in treating diseases caused by SARS-CoV infection, for example, infection caused by SARS-CoV-1 or SARS-CoV-2. In some examples, the interfering RNAs disclosed herein may be used for treating COVID19, a disease caused by SARS-CoV-2.

Accordingly, provided herein are interfering RNAs targeting SARS-CoV, e.g., a particular genomic site within the SARS-CoV genome, pharmaceutical compositions comprising such, and therapeutic uses thereof for inhibiting SARS-CoV infection (e.g., infection by SARS-CoV-1 or SARS-CoV-2) and/or for treating diseases caused by the infection, for example, COVID-19.

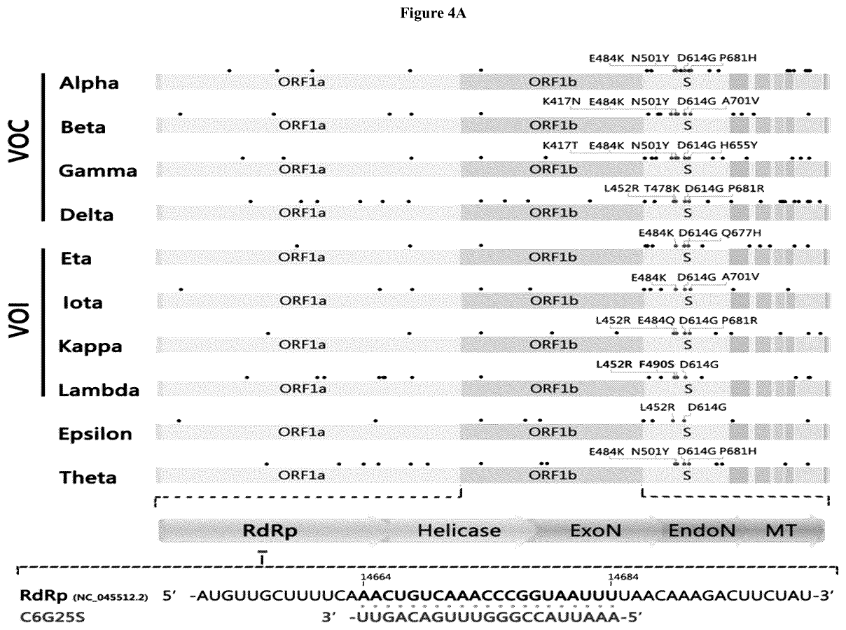

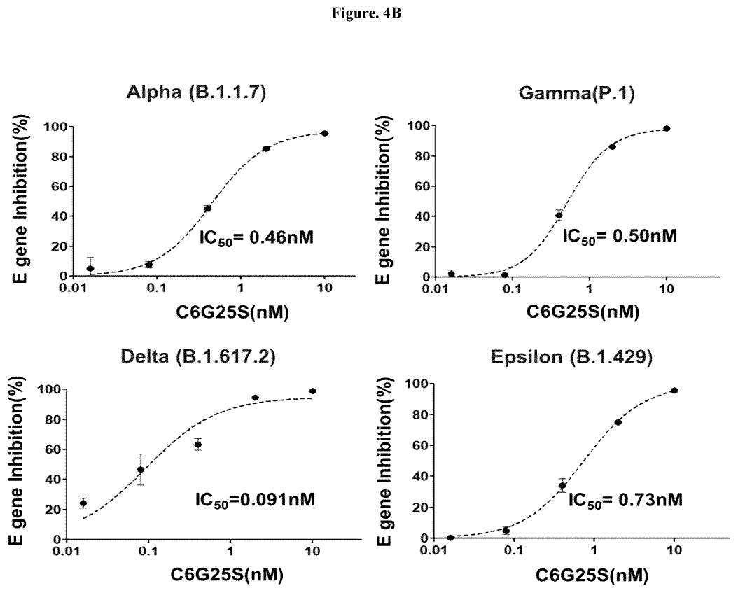

Short-interfering RNA (siRNA), upon entering the cytosol interacts with several proteins to form an RNA-induced silencing complex (RISC) and knocks down the expression of target genes based on sequence complementarity. By targeting a viral genomic site, for example, a highly conserved region of SARS-CoV-2 (e.g., the region within the RdRp gene as shown in FIG. 4A), the siRNAs disclosed herein can inhibit a wide-spectrum of viral variants and, thus could be a one-for-all therapy for the rapidly evolving SARS-CoV-2.

The present disclosure is based, at least in part, on the development of broad-spectrum siRNA molecule that can target a SARS virus such as SARS-CoV-1 or SARS-CoV-2, for example, a highly conserved RdRp region of SARS-CoV-1/2. Such siRNAs show high inhibitory activity against a broad range of SARS-CoV-2 strains (with picomolar IC 50 values), including the most dominate variants (see Example 2 below). Delivery of the siRNAs disclosed herein via a nasal-mediated route, for example, intranasal instillation or aerosol inhalation, showed promising prophylactic and treatment efficacies as observed in an animal model.

Accordingly, provided herein are siRNAs targeting SARS-CoV (e.g., SARS-CoV-1 or SARS-CoV-2), including modified versions, and therapeutic uses thereof for both prophylactic treatment and treatment of actual infections.

### I. Interfering RNAs Targeting SARS-CoV-2

Double-stranded RNA (dsRNA) directs the sequence-specific silencing of mRNA through a process known as RNA interference (RNAi). It has been demonstrated that 21-23 nt fragments of dsRNA are sequence-specific mediators of RNA silencing, e.g., by causing RNA degradation. While not wishing to be bound by theory, it may be that a molecular signal, which may be merely the specific length of the fragments, present in these 21-23 nt fragments recruits cellular factors that mediate RNAi.

Described herein are interfering RNA molecules targeting a SARS-CoV genomic RNA, for example, targeting a specific genomic site therein and methods of using such for inhibiting SARS-CoV replication/production and/or for treating diseases associated with SARS-CoV infection. In some examples, the interfering RNA molecules discloses herein may target a SARS-CoV-1 genomic RNA, for example, targeting a specific genomic site therein and can be used for inhibiting SARS-CoV-1 replication/production and/or for treating diseases associated with SARS-CoV-1 infection. In some examples, the interfering RNA molecules discloses herein may target a SARS-CoV-2 genomic RNA, for example, targeting a specific genomic site therein and can be used for inhibiting SARS-CoV-2 replication/production and/or for treating diseases associated with SARS-CoV-2 infection, for example, COVID19.

As used herein, the term “interfering RNA” refers to any RNA molecule that can be used in inhibiting a target gene, including both mature RNA molecules that are directly involved in RNA interference (e.g., the 21-23nt dsRNA disclosed herein) or a precursor molecule that produces the mature RNA molecule.

An interfering RNA comprises a fragment that is complementary (completely or partially) to a genomic site of SARS-CoV RNA (e.g., SARS-CoV-1 RNA or SARS-CoV-2 RNA). The fragment may be 100% complementary to the target site. Alternatively, the fragment may be partially complementary, e.g., including one or more mismatches but sufficient to form double-strand at the target site to mediate RNA interference.

In some embodiments, an interfering RNA disclosed herein targets a genomic site within the Leader segment of a SARS-CoV RNA (e.g., SARS-CoV-1 RNA or SARS-CoV-2 RNA), for example, targeting a genomic site having the nucleotide sequence of 5′-GAGGCACGUCAACAUCUUA-3′ (SEQ ID NO: 2). Examples include C1 siRNA listed in Table 1.

In some embodiments, an interfering RNA disclosed herein targets a genomic site within the papain-like protease (PLP) gene of a SARS-CoV RNA (e.g., SARS-CoV-1 RNA or SARS-CoV-2 RNA). In some examples, such an interfering RNA may target a genomic site having the nucleotide sequence of 5′-CAGCAUUAAAUCACACUAA-3′ (SEQ ID NO: 4). In some examples, such an interfering RNA may target a genomic site having the nucleotide sequence of 5′-CGGUGUUUAAACCGUGUUU-3′ (SEQ ID NO: 6). Examples include C2 and C3 siRNAs listed in Table 1.

In some embodiments, an interfering RNA disclosed herein targets a genomic site within the 3C-like (3CL) protease gene of a SARS-CoV RNA (e.g., SARS-CoV-1 RNA or SARS-CoV-2 RNA). In some examples, such an interfering RNA may target a genomic site having the nucleotide sequence of 5′-GUGGUACAACUACACUUAA-3′ (SEQ ID NO: 8). In some examples, such an interfering RNA may target a genomic site having the nucleotide sequence of 5′-UGGCUUGAUGACGUAGUUU-3′ (SEQ ID NO: 10). Examples include C4 and C5 siRNAs listed in Table 1.

In some embodiments, an interfering RNA disclosed herein targets a genomic site within the polymerase (POL) gene of a SARS-CoV RNA (also known as RNA-dependent RNA polymerase) (e.g., SARS-CoV-1 POL gene or SARS-CoV-2 POL gene). In some examples, such an interfering RNA may target a genomic site in the nucleotide sequence of 5′-UUGCUUUUCAAACUGUCAAACCCGGUAAUUUUAACAAAGA-3′ (SEQ ID NO: 23). In some instances, the interfering RNA may target a site in the nucleotide sequence of 5′-UUUCAAACUGUCAAACCCGGUAAUUUU-3′ (SEQ ID NO: 24). For example, the interfering RNA may target a site having the nucleotide sequence of 5′-CUGUCAAACCCGGUAAUUU-3′ (SEQ ID NO: 12). In some examples, such an interfering RNA may target a genomic site having the nucleotide sequence of 5′-GCGGUUCACUAUAUGUUAA-3′ (SEQ ID NO: 14). Examples include C6 and C7 siRNAs listed in Table 1.

In some embodiments, an interfering RNA disclosed herein targets a genomic site within the Spike gene of a SARS-CoV RNA (e.g., SARS-CoV-1 RNA or SARS-CoV-2 RNA). In some examples, such an interfering RNA may target a genomic site having the nucleotide sequence of 5′-GCCACUAGUCUCUAGUCAG-3′ (SEQ ID NO: 16). In some examples, such an interfering RNA may target a genomic site having the nucleotide sequence of 5′-CUCCUACUUGGCGUGUUUA-3′ (SEQ ID NO: 18). In some examples, such an interfering RNA may target a genomic site having the nucleotide sequence of 5′-CGCACAUUGCUAACUAAGG-3′ (SEQ ID NO: 20). Examples include C8, C9, and C10 siRNAs listed in Table 1.

In some embodiments, an interfering RNA disclosed herein targets a genomic site within the Envelop gene of a SARS-CoV-2 RNA (e.g., SARS-CoV-1 RNA or SARS-CoV-2 RNA). In some examples, such an interfering RNA may target a genomic site having the nucleotide sequence of 5′-CAGGUACGUUAAUAGUUAA-3′ (SEQ ID NO: 22). Examples include C11 siRNA listed in Table 1.

Nucleotide sequences provided herein, in which modifications are not specifically noted, are meant to encompass both unmodified sequences and modified sequences of any manner.

In some embodiments the interfering RNA discloses herein may be a siRNA, i.e., a double-strand RNA (dsRNA) that contains two separate and complementary RNA chains. Such an siRNA may comprise a sense chain having a nucleotide sequence corresponding to the target genomic site of a SARS-CoV RNA (e.g., SARS-CoV-1 RNA or SARS-CoV-2 RNA) and an antisense chain complementary to the sense chain (and the target genomic site). It would have been known to those skilled in the art that the sense chain and/or the antisense chain does not need to be completely the same or complementary to the target genomic site. One or more mismatches would be allowed as long as the siRNA can still target the genomic site via base-pairing to mediate the RNA interference process. In some instances, the sense chain and/or the antisense chain (whole or a portion thereof) is completely the same or complementary to the target genomic site. In other examples, the interfering RNA discloses here can be a short hairpin RNA (shRNA), which is a RNA molecule forming a tight hairpin structure. Both siRNAs and shRNAs can be designed based on the sequence of the target genomic site in the SARS-CoV RNA (e.g., SARS-CoV-1 RNA or SARS-CoV-2 RNA).

In some embodiments, the interfering RNA disclosed herein (e.g., an siRNA molecule) may contain a sense chain and an antisense chain, which form a double-stranded RNA molecule. In some examples, the sense chain may have 21-23 nucleotides (e.g., 19 nts) and the antisense chain may have 23-25 nucleotides (e.g., 23 nts) with two nucleotide overhang at its 3′ end (—N₁N₂-3′) relative to the sense chain. The overhang nucleotides can be any of A, G, C, and U. In some instances, the 3′ end nucleotide (N₂) may be U. Alternatively or in addition, N₁ may be complementary to the corresponding nucleotide at the targeting site. In some examples, the 3′ end nucleotide of the sense chain and the 5′-end nucleotide of the antisense chain may form a base pair, e.g., A/U pair or G/C pair.

In some embodiments, the anti-SARS-CoV-2 interfering RNA disclosed herein can be an siRNA molecule, for example, those listed in Table 1 below. In specific examples, the siRNA is one of C6, C7, C8, C10, or C11.

In some examples, the siRNA may comprise a sense chain comprising 5′-GAGGCACGUCAACAUCUUX₁-3′ (SEQ ID NO: 25) and an antisense chain comprising 5′-X₂AAGAUGUUGACGUGCCUCN₁N₂-3′ (SEQ ID NO: 26). X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-GAGGCACGUCAACAUCUUX₁-3′ (SEQ ID NO: 25) and an antisense chain comprising 5′-X₂AAGAUGUUGACGUGCCUCUU-3,′ (SEQ ID NO: 47) in which X₁ and X₂ for an A/U pair.

In some examples, the siRNA may comprise a sense chain comprising 5′-CAGCAUUAAAUCACACUAX₁-3′ (SEQ ID NO: 27) and an antisense chain comprising 5′-X₂UAGUGUGAUUUAAUGCUGN₁N₂-3.′ (SEQ ID NO: 28) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-CAGCAUUAAAUCACACUAX₁-3′ (SEQ ID NO: 27) and an antisense chain comprising 5′-X₂UAGUGUGAUUUAAUGCUGUU-3′, (SEQ ID NO: 48) in which X₁ and X₂ for an A/U pair.

In some examples, the siRNA may comprise a sense chain comprising 5′-CGGUGUUUAAACCGUGUUX₁-3′(SEQ ID NO: 29) and an antisense chain comprising 5′-X₂AACACGGUUUAAACACCGN₁N2-3.′ (SEQ ID NO: 30) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-CGGUGUUUAAACCGUGUUX₁-3′(SEQ ID NO: 29) and an antisense chain comprising 5′-X₂AACACGGUUUAAACACCGUU-3′ (SEQ ID NO: 49), in which X₁ and X₂ for an A/U pair.

In some examples, the siRNA may comprise a sense chain comprising 5′-GUGGUACAACUACACUUAX₁-3′ (SEQ ID NO: 31), and an antisense chain comprising 5′-X₂UAAGUGUAGUUGUACCACN₁N2-3.′ (SEQ ID NO: 32) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-GUGGUACAACUACACUUAX₁-3′ (SEQ ID NO: 31), and an antisense chain comprising 5′-X₂UAAGUGUAGUUGUACCACUU-3′(SEQ ID NO: 50), in which X₁ and X₂ for an A/U pair.

In some examples, the siRNA may comprise a sense chain comprising 5′-UGGCUUGAUGACGUAGUUX₁-3′ (SEQ ID NO: 33), and an antisense chain comprising 5′-X₂AACUACGUCAUCAAGCCAN₁N2-3.′ (SEQ ID NO: 34) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-UGGCUUGAUGACGUAGUUX₁-3′ (SEQ ID NO: 33), and an antisense chain comprising 5′-X₂AACUACGUCAUCAAGCCAUU-3′ (SEQ ID NO: 51), in which X₁ and X₂ for an A/U pair.

In some examples, the siRNA may comprise a sense chain comprising 5′-CUGUCAAACCCGGUAAUUX₁-3′ (SEQ ID NO: 35), and an antisense chain comprising 5′-X₂AAUUACCGGGUUUGACAGN₁N2-3.′ (SEQ ID NO: 36) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-CUGUCAAACCCGGUAAUUX₁-3′ (SEQ ID NO:_35), and an antisense chain comprising 5′-X₂AAUUACCGGGUUUGACAGUU-3′ (SEQ ID NO:_52), in which X₁ and X₂ for an A/U pair.

In some examples, the siRNA may comprise a sense chain comprising 5′-GCGGUUCACUAUAUGUUAX₁-3′ (SEQ ID NO:_37), and an antisense chain comprising 5′-X₂UAACAUAUAGUGAACCGCN₁N2-3.′ (SEQ ID NO:_38) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-GCGGUUCACUAUAUGUUAX₁-3′ (SEQ ID NO:_37), and an antisense chain comprising 5′-X₂UAACAUAUAGUGAACCGCUU-3′ (SEQ ID NO:_53), in which X₁ and X₂ for an A/U pair.

In some examples, the siRNA may comprise a sense chain comprising 5′-GCCACUAGUCUCUAGUCAX₁-3′ (SEQ ID NO:_39), and an antisense chain comprising 5′-X₂UGACUAGAGACUAGUGGCN₁N2-3.′ (SEQ ID NO:_40) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-GCCACUAGUCUCUAGUCAX₁-3′ (SEQ ID NO:_39), and an antisense chain comprising 5′-X₂UGACUAGAGACUAGUGGCUU-3′ (SEQ ID NO:_54), in which X₁ and X₂ for an A/U pair.

In some examples, the siRNA may comprise a sense chain comprising 5′-CUCCUACUUGGCGUGUUUX₁-3′ (SEQ ID NO: 41), and an antisense chain comprising 5′-X₂AAACACGCCAAGUAGGAGN₁N2-3.′ (SEQ ID NO: 42) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-CUCCUACUUGGCGUGUUUX₁-3′ (SEQ ID NO: 41), and an antisense chain comprising 5′-X₂AAACACGCCAAGUAGGAGUU-3′ (SEQ ID NO: 62), in which X₁ and X₂ for an A/U pair.

In some examples, the siRNA may comprise a sense chain comprising 5′-CGCACAUUGCUAACUAAGX₁-3′ (SEQ ID NO: 43), and an antisense chain comprising 5′-X₂CUUAGUUAGCAAUGUGCGN₁N2-3.′ (SEQ ID NO: 44) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In specific examples, the siRNA may comprise a sense chain comprising 5′-CGCACAUUGCUAACUAAGX₁-3′ (SEQ ID NO: 43), and an antisense chain comprising 5′-X₂CUUAGUUAGCAAUGUGCGUU-3′ (SEQ ID NO:_56), in which X₁ and X₂ for an A/U pair.

In some examples, the siRNA may comprise a sense chain comprising 5′-CAGGUACGUUAAUAGUUAX₁-3′ (SEQ ID NO: 45), and an antisense chain comprising 5′-X₂UAACUAUUAACGUACCUGN₁N2-3.′ (SEQ ID NO: 46) X₁ and X₂ form an A/U or G/C base pair. Each of N1 and N2 can be any nucleotide (A, G, C, or U). In some instances, X₁ and X₂ form an A/U pair. Alternative or in addition, N₂ is U and N1 is complementary to the corresponding nucleotide at the targeting site. In some examples, the siRNA may comprise a sense chain comprising 5′-CAGGUACGUUAAUAGUUAX₁-3′ (SEQ ID NO: 45), and an antisense chain comprising 5′-X₂UAACUAUUAACGUACCUGUU-3′ (SEQ ID NO: 57), in which X₁ and X₂ for an A/U pair.

In some instances, the siRNA disclosed herein may comprise the same sense chain and/or same antisense chain as C6, C7, C8, C10, or C11. In other instances, the siRNA disclosed herein may comprise a sense chain that is at least 80% (e.g., at least 85%, at least 90%, at least 95%, or higher) identical to the sense chain of C6 and/or comprise an antisense chain that is at least 80% (e.g., at least 85%, at least 90%, at least 95%, or higher) identical to the sense chain of C6. In other instances, the siRNA disclosed herein may comprise a sense chain that is at least 80% (e.g., at least 85%, at least 90%, at least 95%, or higher) identical to the sense chain of C7 and/or comprise an antisense chain that is at least 80% (e.g., at least 85%, at least 90%, at least 95%, or higher) identical to the sense chain of C7. In other instances, the siRNA disclosed herein may comprise a sense chain that is at least 80% (e.g., at least 85%, at least 90%, at least 95%, or higher) identical to the sense chain of C8 and/or comprise an antisense chain that is at least 80% (e.g., at least 85%, at least 90%, at least 95%, or higher) identical to the sense chain of C8. In other instances, the siRNA disclosed herein may comprise a sense chain that is at least 80% (e.g., at least 85%, at least 90%, at least 95%, or higher) identical to the sense chain of C10 and/or comprise an antisense chain that is at least 80% (e.g., at least 85%, at least 90%, at least 95%, or higher) identical to the sense chain of C10. In other instances, the siRNA disclosed herein may comprise a sense chain that is at least 80% (e.g., at least 85%, at least 90%, at least 95%, or higher) identical to the sense chain of C11 and/or comprise an antisense chain that is at least 80% (e.g., at least 85%, at least 90%, at least 95%, or higher) identical to the sense chain of C11.

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-SARS-CoV2 siRNA described herein may contain up to 6 (e.g., up to 6, 5, 4, 3, or 2) nucleotide variations as compared with the sense chain and antisense chain (collectively or separately) of a reference siRNA, such as those listed in Table 1, for example, C6, C7, C8, C10, or C11.

In some embodiments, any of the anti-SARS-CoV-2 interfering RNAs (e.g., siRNAs such as C6, C7, C8, C10, or C11) described herein may contain non-naturally-occurring nucleobases, sugars, or covalent internucleoside linkages (backbones). Such a modified oligonucleotide confers desirable properties, for example, enhanced cellular uptake, improved affinity to the target nucleic acid, increased in vivo stability, enhance in vivo stability (e.g., resistant to nuclease degradation), and/or reduce immunogenicity.

In one example, the anti-SARS-CoV-2 interfering RNAs (e.g., siRNAs such as C6, C7, C8, C10, or C11) described herein has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the anti-SARS-CoV-2 interfering RNAs (e.g., siRNAs such as C6, C7, C8, C10, or C11) described herein include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, the anti-SARS-CoV-2 interfering RNAs (e.g., siRNAs such as C6, C7, C8, C10, or C11) described herein include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y S., Chapter 15, Antisense Research and Applications, pages 289-302, CRC Press, 1993. Certain of these nucleobases are particularly useful for increasing the binding affinity of the interfering RNA molecules to their targeting sites. These include 5-substituted pyrimidines, 6-azapyrimidines and N-2, N-6 and 0-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Alternatively or in addition, the anti-SARS-CoV-2 interfering RNAs (e.g., siRNAs such as C6, C7, C8, C10, or C11) as described herein may comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form duplexes. LNA nucleotides can be used in any of the anti-SARS-CoV-2 interfering RNAs (e.g., siRNAs such as C6, C7, C8, C10, or C11) described herein. In some examples, up to 50% (e.g., 40%, 30%, 20%, or 10%) of the nucleotides in an interfering RNA are LNAs.

In some embodiments, any of the anti-SARS-CoV-2 interfering RNAs (e.g., siRNAs such as C6, C7, C8, C10, or C11) described herein may be conjugated to a ligand or encapsulated into vesicles that can facilitate the delivery of siRNA to desired cells/tissues and/or facilitate cellular uptake. Suitable ligands include, but are not limited to, carbohydrate, peptide, antibody, polymer, small molecule, cholesterol and aptamer.

Any of the anti-SARS-CoV-2 interfering RNAs (e.g., siRNAs such as C6, C7, C8, C10, or C11) described herein can be prepared by conventional methods, e.g., chemical synthesis or in vitro transcription. Their intended bioactivity as described herein can be verified by, e.g., those described in the Examples below. Vectors for expressing any of the anti-SARS-CoV-2 interfering RNAs (e.g., siRNAs such as C6, C7, C8, C10, or C11) are also within the scope of the present disclosure.

In specific examples, the siRNA disclosed herein for use in treating SARS-CoV-2 infection (prophylactic or actual treatment) is a modified siRNA of C6G25S (see Table 4 below). In other examples, the siRNA disclosed herein for use in treating SARS-CoV-2 infection (prophylactic or actual treatment) is a modified siRNA of C8G25S (see Table 4 below). In yet other examples, the siRNA disclosed herein for use in treating SARS-CoV-2 infection (prophylactic or actual treatment) is a modified siRNA of C10G31A (see Table 4 below).

### II. Pharmaceutical Compositions

Any of the interfering RNAs (e.g., siRNAs such as C6, C7, C8, C10, and C11 as disclosed herein, unmodified or modified such as C6G25S, C8G25S, or C10G31A) may be formulated into a suitable pharmaceutical composition. The pharmaceutical compositions as described herein can further comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some examples, the pharmaceutical composition described herein includes pulmonary compatible excipients. Suitable such excipients include, but not limited to, richloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane, purified water, ethanol, propylene glycol, glycerin, PEG (e.g., PEG400, PEG 600, PEG 800 and PEG 1000), sorbitan trioleate, soya lecithin, lecithin, oleic acid, Polysorbate 80, magnesium stearate and sodium laury sulfate, methylparaben, propylparaben, chlorobutanol, benzalkonium chloride, cetylpyridinium chloride, thymol, ascorbic acid, sodium bisulfite, sodium metabisulfite, EDTA, sodium hydroxide, tromethamine, ammonia, HCl, H₂SO₄, HNO₃, citric acid, CaCl₂, CaCO₃, sodium citrate, sodium chloride, disodium EDTA, saccharin, menthol, ascorbic acid, glycine, lysine, gelatin, povidone K25, silicon dioxide, titanium dioxide, zinc oxide, lactose, lactose monohydrate, lactose anhydrate, mannitol, and dextrose.

In other examples, the pharmaceutical composition described herein can be formulated in sustained-release format. Suitable examples of sustained-release preparations include semipermeable matrices of solid hydrophobic polymers which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle or a sealed container to be manually accessed.

The pharmaceutical compositions described herein can be in unit dosage forms such as solids, solutions or suspensions, or suppositories, for administration by inhalation or insufflation, intrathecal, intrapulmonary or intracerebral routes, oral, parenteral or rectal administration.

For preparing solid compositions, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as powder collections, tablets, pills and capsules. This solid preformulation composition is then subdivided into unit dosage forms of the type described above containing a suitable amount of the active ingredient in the composition.

Suitable surface-active agents include, in particular, non-ionic agents, such as polyoxyethylenesorbitans (e.g., TWEEN® 20, 40, 60, 80 or 85) and other sorbitans (e.g., SPAN® 20, 40, 60, 80 or 85). Compositions with a surface-active agent will conveniently comprise between 0.05 and 5% surface-active agent, for example, between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™, and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. In some embodiments, the compositions are composed of particle sized between 10 nm to 100 mm.

Compositions in preferably sterile pharmaceutically acceptable solvents may be nebulized by use of gases. Nebulized solutions may be breathed directly from the nebulizing device or the nebulizing device may be attached to a face mask, tent, endotracheal tube and/or intermittent positive pressure breathing machine (ventilator). Solution, suspension or powder compositions may be administered, preferably orally or nasally, from devices which deliver the formulation in an appropriate manner.

In some instances, compositions comprising any of the siRNAs disclosed herein may be formulated for nasal spray (e.g., aerosol inhalation) or for intranasal delivery.

In some embodiments, any of the anti-SARS-CoV-2 interfering RNAs (e.g., siRNA such as C6, C7, C8, C10, or C11, unmodified or modified such as C6G25S, C8G25S, or C10G31A) can be encapsulated or attached to a liposome, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, any of the anti-SARS-CoV-2 interfering RNAs (e.g., siRNA such as C6, C7, C8, C10, or C11, unmodified or modified such as C6G25S, C8G25S, or C10G31A) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

Any of the pharmaceutical compositions comprising the anti-SARS-CoV interfering RNAs (e.g., anti-SARS-CoV-1 interfering RNAs or anti-SARS-CoV-2 interfering RNAs), disclosed herein, may further comprise a component that enhances transport of the composition from endosomes and/or lysosomes to cytoplasm. Examples include a pH-sensitive agent (e.g., a pH-sensitive peptide).

In some embodiments, any of the pharmaceutical compositions herein may further comprise a second therapeutic agent based on the intended therapeutic uses of the composition.

### III. Therapeutic Applications

In some aspects, the present disclosure provides uses of any of the interfering RNAs (e.g., siRNAs such as C6, C7, C8, C10, or C11, unmodified or modified such as C6G25S, C8G25S, or C10G31A) disclosed herein or any of the compositions comprising such for inhibiting, treating, reducing the viral load, and/or reducing morbidity or mortality in the clinical outcomes, in patients suffering from a coronavirus infection, for example, suffering COVID-19. In yet another aspect, the present disclosure further provides methods of reducing the risk that an individual will develop a pathological coronavirus infection that has clinical sequelae. The methods generally involve administering a therapeutically effective amount of a therapeutically effective amount of the composition herein.

COVID-19 is a disease caused by severe acute respiratory syndrome coronavirus 2 (SARS-CoV-2), previously known as 2019 novel coronavirus. In other instances, the patient may have an infection caused by another coronavirus such as severe acute respiratory syndrome coronavirus (SARS-CoV), for example, SARS-CoV-1.

Any of the anti-SARS-CoV-2 interfering RNA (e.g., siRNAs such as C6, C7, C8, C10, or C11, unmodified or modified such as C6G25S, C8G25S, or C10G31A) disclosed herein can be used to inhibit SARS-CoV-2 replication and production, thereby effective in suppressing viral infection and treating diseases or disorders caused by coronavirus infection, for example, COVID-19.

To practice the method disclosed herein, an effective amount of the pharmaceutical composition described herein that contains at least one anti-SARS-CoV-2 interfering RNA (e.g., siRNAs such as C6, C7, C8, C10, or C11, unmodified or modified such as C6G25S, C8G25S, or C10G31A) can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the anti-SARS-CoV interfering RNA-containing composition as described herein can be aerosolized using a fluorocarbon formulation and a metered dose inhaler, or inhaled as a lyophilized and milled powder.

As used herein, “an effective amount” refers to the amount of each active agent required to confer therapeutic effect on the subject, either alone or in combination with one or more other active agents. In some embodiments, the therapeutic effect is reduced SARS-CoV virus (e.g., SARS-CoV-1 or SARS-CoV-2) replication and/or production. Determination of whether an amount of the anti-SARS-CoV-1 or anti-SARS-CoV-2 interfering RNA achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of a interfering RNA (e.g., siRNAs such as C6, C7, C8, C10, or C11, unmodified or modified such as C6G25S, C8G25S, or C10G31A) may be appropriate. Various formulations and devices for achieving sustained release are known in the art.

In one example, dosages for an anti-SARS-CoV-2 interfering RNA (e.g., siRNAs such as C6, C7, C8, C10, or C11, unmodified or modified such as C6G25S, C8G25S, or C10G31A) as described herein may be determined empirically in individuals who have been given one or more administration(s) of the anti-SARS-CoV-2 interfering RNA. Individuals are given incremental dosages of the antagonist. To assess efficacy of the antagonist, an indicator of the disease/disorder can be followed.

Generally, for administration of any of the anti-SARS-CoV-2 interfering RNA (e.g., siRNAs such as C6, C7, C8, C10, or C11, unmodified or modified such as C6G25S, C8G25S, or C10G31A) described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the anti-SARS-CoV-2 interfering RNA (e.g., siRNAs such as C6, C7, C8, C10, or C11), or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the anti-SARS-CoV-2 interfering RNAs used (e.g., siRNAs such as C6, C7, C8, C10, or C11)) can vary over time.

In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an anti-SARS-CoV-2 interfering RNA (e.g., siRNA such as C6, C7, C8, C10, or C11, unmodified or modified such as C6G25S, C8G25S, or C10G31A) as described herein will depend on the specific siRNA, the type and severity of the disease/disorder, whether the siRNA is administered for preventive or therapeutic purposes, previous therapy, the patient's clinical history and response to the antagonist, and the discretion of the attending physician. A clinician may administer an anti-SARS-CoV-2 interfering RNA (e.g., siRNA such as C6, C7, C8, C10, or C11, unmodified or modified such as C6G25S, C8G25S, or C10G31A), until a dosage is reached that achieves the desired result. In some embodiments, the desired result is a decrease in tumor burden, a decrease in cancer cells, or increased immune activity. Methods of determining whether a dosage resulted in the desired result would be evident to one of skill in the art. Administration of one or more anti-SARS-CoV-2 interfering RNAs (e.g., siRNAs such as C6, C7, C8, C10, or C11, unmodified or modified such as C6G25S, C8G25S, or C10G31A) can be continuous or intermittent, depending, for example, upon the recipient's physiological condition, whether the purpose of the administration is therapeutic or prophylactic, and other factors known to skilled practitioners. The administration of an anti-SARS-CoV-2 interfering RNA (e.g., siRNA such as C6, C7, C8, C10, or C11, unmodified or modified such as C6G25S, C8G25S, or C10G31A) may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

To achieve any of the intended therapeutic effects described herein, an effective amount of a composition herein may be administered to a subject in need of the treatment via a suitable route.

As used herein, “an effective amount” refers to the amount of each active agent required to confer therapeutic effect on the subject, either alone or in combination with one or more other active agents, such as one or more of the second therapeutic agents described herein. In some embodiments, the therapeutic effect is improvement of basic conditions of virus infection. In some embodiments, the therapeutic effect is alleviating one or more symptoms associated with any of the infection by the virus described herein.

Determination of whether an amount of the composition as described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration, genetic factors and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration and/or route of administration may be determined and adjusted over the course of therapy, and is generally, but not necessarily, based on treatment and/or suppression and/or amelioration and/or delay of a target disease/disorder. Alternatively, sustained continuous release formulations of a composition as described herein may be appropriate. Various formulations and devices for achieving sustained release are known in the art.

In some embodiments, the effective amount can be a prophylactically effective amount (e.g., amount effective for inhibiting, treating, reducing the viral load, and/or reducing morbidity or mortality in a subject suffering from the viral infection in need of such an effect) to reduce the risk of having coronarivus infection.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some embodiments, the composition can be administered via a nasal route, for example, intranasal spray, nasal spray, or nasal drops. In specific examples, the composition can be administered to a subject in need of the treatment (e.g., prophylactic or actual) via both intranasal instillation and aerosol inhalation.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble anti-SARS-CoV-2 interfering RNAs (e.g., siRNAs such as C6, C7, C8, C10, or C11) can be administered by the drip method, whereby a pharmaceutical formulation containing the interfering RNA and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the siRNA disclosed herein, can be dissolved and administered in a pharmaceutical excipient such as Water-for-Injection, 0.9% saline, or 5% glucose solution.

In one embodiment, an anti-SARS-CoV-2 interfering RNA (e.g., siRNA such as C6, C7, C8, C10, or C11) is administered via site-specific or targeted local delivery techniques. Examples of site-specific or targeted local delivery techniques include various implantable depot sources of the siRNA or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing a polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

In some embodiments, any of the anti-SARS-CoV-2 interfering RNAs (e.g., siRNAs such as C6, C7, C8, C10, or C11, unmodified or modified such as C6G25S, C8G25S, or C10G31A) or a pharmaceutical composition comprising such can be administered by pulmonary delivery system, that is, the active pharmaceutical ingredient is administered into lung. The pulmonary delivery system can be an inhaler system. In some embodiment, the inhaler system is a pressurized metered dose inhaler, a dry powder inhaler, or a nebulizer. In some embodiment, the inhaler system is with a spacer.

In some embodiment, the pressurized metered dose inhaler includes a propellent, a co-solvent, and/or a surfactant. In some embodiment, the propellent is selected from the group comprising of fluorinated hydrocarbons such as trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane. In some embodiment, the co-solvent is selected from the group comprising of purified water, ethanol, propylene glycol, glycerin, PEG400, PEG 600, PEG 800 and PEG 1000. In some embodiment, the surfactant or lubricants is selected from the group comprising of sorbitan trioleate, soya lecithin, lecithin, oleic acid, Polysorbate 80, magnesium stearate and sodium laury sulfate. In some embodiment, the preservatives or antioxidants is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some embodiment, the pH adjustments or tonicity adjustments is selected from the group comprising of sodium oxide, tromethamine, ammonia, HCl, H₂SO₄, HNO₃, citric acid, CaCl₂, CaCO₃.

In some embodiment, the dry powder inhaler includes a disperse agent. In some embodiment, the disperse agent or carrier particle is selected from the group comprising of lactose, lactose monohydrate, lactose anhydrate, mannitol, dextrose which their particle size is about 1-100 μm.

In some embodiment, the nebulizer may include a co-solvent, a surfactant, lubricant, preservative and/or antioxidant. In some embodiment, the co-solvent is selected from the group comprising of purified water, ethanol, propylene glycol, glycerin, PEG (e.g., PEG400, PEG600, PEG800 and/or PEG 1000). In some examples, the surfactant or lubricant is selected from the group comprising of sorbitan trioleate, soya lecithin, lecithin, oleic acid, magnesium stearate and sodium laury sulfate. In some examples, the preservative or antioxidant is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some examples, the nebulizer further includes a pH adjustment or a tonicity adjustment, which is selected from the group comprising of sodium oxide, tromethamine, ammonia, HCl, H₂SO₄, HNO₃, citric acid, CaCl₂, CaCO₃.

Therapeutic compositions containing a polynucleotide (e.g., the anti-SARS-CoV-2 siRNAs described herein or vectors for producing such) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The term “about” or “approximately” used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, etc. A human subject who needs the treatment may be a human patient having, at risk for, or suspected of having a target disease/disorder, such as an infection by virus, or by Coronavirus. In some embodiments, the subject (e.g., a human patient) may have or be suspected of having infection by a coronavirus. In some examples, the subject is a human patient having or suspected of having infection by SARS-CoV-1. In some examples, the subject is a human patient having or suspected of having infection by SARS-CoV-2. In some examples, the subject is a human patient having or suspected of having COVID-19.

Treatment efficacy for a target disease/disorder can be assessed by methods well-known in the art.

### IV. Combined Therapy

Also provided herein are combined therapies using any of the compositions described herein, comprising one or more of the anti-SARS-CoV interfering RNA (e.g., siRNA such as C6, C7, C8, C10, or C11, unmodified or modified such as C6G25S, C8G25S, or C10G31A) and a second therapeutic agent, such as those described herein. The term combination therapy, as used herein, embraces administration of these agents (e.g., the anti-SARS-CoV interfering RNA and an antiviral agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route including, but not limited to, oral routes, intravenous routes, intramuscular, subcutaneous routes, direct absorption through mucous membrane tissues, and pulmonary delivery routes. The agents can be administered by the same route or by different routes. For example, a first agent (e.g., a composition described herein) can be administered by pulmonary delivery routes, and a second agent (e.g., an antiviral agent) can be administered intravenously.

Examples of the additional pharmaceutical agent selected from a viral entry inhibitor, a viral uncoating inhibitor, a viral reverse transcriptase inhibitor, a viral protein synthesis inhibitor, a viral protease inhibitor, a viral polymerase inhibitor, a viral integrase inhibitor, an interferon, or the combination thereof. Examples of viral entry inhibitor include, but is not limited to, maraviroc, enfuvirtide, ibalizumab, fostemsavir, plerixafor, epigallocatechin gallate, vicriviroc, aplaviroc, maraviroc, tromantadine, nitazoxanide, umifenovir, and podofilox. Examples of viral uncoating inhibitor include, but not limited to, amantadine, rimantadine, and pleconaril. Examples of reverse transcriptase inhibitor include, but not limited to zidovudine, didanosine, zalcitabine, stavudine, lamivudine, abacavir, emtricitabine, entecavir, truvada, nevirapine, raltegravir, and tenofovir disoproxil. Examples of viral protease inhibitor, include but not limited to, fosamprenavir, ritonavir, atazanavir, nelfinavir, indinavir, saquinavir, saquinavir, famciclovir, fomivirsen, lopinavir, ribavirin, darunavir, oseltamivir, and tipranavir. Examples of viral polymerase inhibitor, include but not limited to, amatoxins, rifamycin, cytarabine, fidaxomicin, tagetitoxin, foscarnet sodium, idoxuridine, penciclovir, sofosbuvir, trifluridine, valacyclovir, valganciclovir, vidarabine, and remdesivir. Examples of viral integrase inhibitor, include but not limited to, raltegarvir, elvitegravir, dolutegravir, bictegravir, and cabotegravir. Examples of interferon, include but not limited to, type I interferon, type II interferon, type III interferon, and peginterferon alfa-2a.

In some examples, the additional therapeutic agent may comprise one or more anti-SARS-CoV-2 antibody, for example, REGN10933 and REGN10987. In other examples, the additional therapeutic agent may be a small molecule anti-SARS agent, such as remdesivir. In yet other examples, the additional therapeutic agent may comprise a steroid compound such as a corticosteroid (e.g., dexamethasone, hydrocortisone, or methylprednisolone).

As used herein, the term “sequential” means, unless otherwise specified, characterized by a regular sequence or order, e.g., if a dosage regimen includes the administration of a composition and an antiviral agent, a sequential dosage regimen could include administration of the composition before, simultaneously, substantially simultaneously, or after administration of the antiviral agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the agents described herein.

Combination therapy can also embrace the administration of the agents described herein (e.g., the composition and an antiviral agent) in further combination with other biologically active ingredients (e.g., a different antiviral agent) and non-drug therapies.

It should be appreciated that any combination of a composition described herein and a second therapeutic agent (e.g., an antiviral agent) may be used in any sequence for treating a target disease. The combinations described herein may be selected on the basis of a number of factors, which include but are not limited to the effectiveness of inhibiting virus or at least one symptom associated with the virus infection.

### V. Kits for Treating Coronavirus Infection

The present disclosure also provides kits for use in treating coronavirus infection. Such kits can include one or more containers comprising the anti-SARS-CoV-2 interfering RNA (e.g., siRNA such as C6, C7, C8, C10, or C11, unmodified or modified such as C6G25S, C8G25S, or C10G31A) or a composition comprising such as described herein and optionally one or more of the second therapeutic agents as also described herein.

In some embodiments, the kit can comprise instructions for use in accordance with any of the methods described herein. The included instructions can comprise, for example, a description of administration of the siRNA compound and optionally a description of administration of the second therapeutic agent(s) to improve medical conditions of virus infection or in the rick of virus infection. The kit may further comprise a description of selecting an individual suitable for treatment based on identifying whether that individual has the disease or is at risk for the disease. In still other embodiments, the instructions comprise a description of administering one or more agents of the disclosure to an individual at risk of virus infection.

The instructions relating to the use of the siRNA compound to achieve the intended therapeutic effects generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk, or QR code) are also acceptable.

The label or package insert may indicate that the composition is used for the intended therapeutic utilities. Instructions may be provided for practicing any of the methods described herein.

The kits of this invention are in suitable packaging. Suitable packaging includes, but is not limited to, chambers, vials, bottles, jars, flexible packaging (e.g., sealed Mylar or plastic bags), and the like. Also contemplated are packages for use in combination with a specific device, such as an inhaler, nebulizer, ventilator, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Inhibition of SARS-CoV-2 by Exemplary siRNAs Targeting SARS-CoV-2

The outbreak of coronavirus disease (COVID-19) has rapidly evolved into a worldwide pandemic with a substantial health and economic burden. COVID-19 disease is caused by the severe respirator syndrome coronavirus 2 (SARS-CoV-2), belonging to the coronaviruses (CoV) family. The RNA genome of SARS-CoV-2 is a non-segmented positive-sense RNA with an average size of 30 kb. Two-thirds of the genome at its 5′ end encodes two polyproteins that contain 16 nonstructural proteins critical for viral replication. One-third of the genome at its 3′ end encodes 4 structural proteins and some accessory proteins.

RNA interference (RNAi) is mediated by an RNA-induced silencing complex (RISC) that identifies and retains the antisense strand of double-stranded siRNA and destroys the complimentary mRNA target. Therefore, RNAi is a suitable strategy to destroy viral RNA genome and inhibit RNA virus replication and the expression of viral proteins.

There were 29771 full-length SARS-CoV-2 genome sequences in the GenBank database, as of June 2020, and these were analyzed for 19-nucleotide stretches that showed at least 99% identity (high conservation) in the SARS-CoV-2 genome. The RNA secondary structure of SARS-CoV-2 genome was evaluated because RNA target accessibility affects the siRNA efficacy. The design and selection of siRNA candidates was performed considering the following criteria: (1) target the RNA regions with weak or no RNA secondary structure; (2) low off-target possibility: low cross-reactivity to human mRNA database; and (3) low number of essential genes predicted to be targeted by the siRNA candidates. The siRNA candidates were selected from top sequences with high coverage in the SARS-CoV-2 genomes, low off-target rate, and low propensity for RNA secondary structure.

Eleven top siRNA candidates were selected for further in vitro screening experiments. Sequences of these siRNA are listed below:

TABLE 1
Sequences of siRNA Candidates
Targeting SARS-COV-2

| | | | SEQ |
|---|---|---|---|
| siRNA | | | ID |
| Candidate | Strand | Sequence 5′-3′ | NOS |
| C1 | Antisense | UAAGAUGUUGACGUGCCUCUU | 1 |
| | Sense | GAGGCACGUCAACAUCUUA | 2 |
| C2 | Antisense | UUAGUGUGAUUUAAUGCUGUU | 3 |
| | Sense | CAGCAUUAAAUCACACUAA | 4 |
| C3 | Antisense | AAACACGGUUUAAACACCGUU | 5 |
| | Sense | CGGUGUUUAAACCGUGUUU | 6 |
| C4 | Antisense | UUAAGUGUAGUUGUACCACUU | 7 |
| | Sense | GUGGUACAACUACACUUAA | 8 |
| C5 | Antisense | AAACUACGUCAUCAAGCCAUU | 9 |
| | Sense | UGGCUUGAUGACGUAGUUU | 10 |
| C6 | Antisense | AAAUUACCGGGUUUGACAGUU | 11 |
| | Sense | CUGUCAAACCCGGUAAUUU | 12 |
| C7 | Antisense | UUAACAUAUAGUGAACCGCUU | 13 |
| | Sense | GCGGUUCACUAUAUGUUAA | 14 |
| C8 | Antisense | UUGACUAGAGACUAGUGGCUU | 15 |
| | Sense | GCCACUAGUCUCUAGUCAA | 74 |
| C9 | Antisense | UAAACACGCCAAGUAGGAGUU | 17 |
| | Sense | CUCCUACUUGGCGUGUUUA | 18 |
| C10 | Antisense | UCUUAGUUAGCAAUGUGCGUU | 19 |
| | Sense | CGCACAUUGCUAACUAAGA | 75 |
| C11 | Antisense | UUAACUAUUAACGUACCUGUU | 21 |
| | Sense | CAGGUACGUUAAUAGUUAA | 22 |

The 11 siRNA candidates listed in Table 1 were synthesized and screened in Vero cells for their inhibitory activity against SARS-CoV-2. Briefly, the Vero cells were reverse-transfected with siRNA candidates and then seeded into 24-well culture plates. At 24 hr post-transfection, the siRNA-transfected cells were infected with SARS-CoV-2 virus. After 24 hr infection, total RNAs were isolated from virus-infected Vero cells. The knockdown of SARS-CoV-2 RNA genome by siRNAs was determined by RT-qPCR targeting the E (envelope) gene. Virus titers in the culture medium were quantified by a plaque assay. A brief description of each assay is provided below.

RT-qPCR

Total cellular RNA was extracted with a NucleoSpin RNA mini kit (Macherey-Nagel, Duren). The amount of viral RNA was determined by reverse transcription-quantitative polymerase chain reaction (RT-qPCR) of viral E gene on a QuantStudio 5 Real-Time PCR System (Applied Biosystems) using an iTaq Universal Probes One-Step RT-PCR Kit (Bio-Rad). The primers and probe targeting the SARS-CoV-2 were as follows:

forward primer,
(SEQ ID NO: 62)
5′-ACAGGTACGTTAATAGTTAATAGCGT-3′;
reverse primer,
(SEQ ID NO: 63)
5′-ACATTGCAGCAGTACGCACACA-3′;
and
probe,
(SEQ ID NO: 64)
5′-ACACTAGCCATCCTTACTGCGCTTCG-3′

Plasmid containing partial E fragment was used as a standard to calculate the viral load (copies/μl).

Plaque Assay

Vero cells were seeded in 24-well culture plates in DMEM supplemented with 10% FBS and grown to confluent monolayers for 24 hr. Cells were washed with PBS and inoculated with serial 10-fold dilutions of virus-containing medium in triplicates. After 1 hr of adsorption, the virus supernatant was removed. Cells were then washed with PBS, overlaid with medium containing 1% methylcellulose and incubated for 3-5 days. The plate was subsequently fixed with 10% formaldehyde in PBS for 1 hr, washed to remove the overlay medium and stained with 0.7% crystal violet. Plaques were counted to calculate the virus titers expressed as PFU/ml.

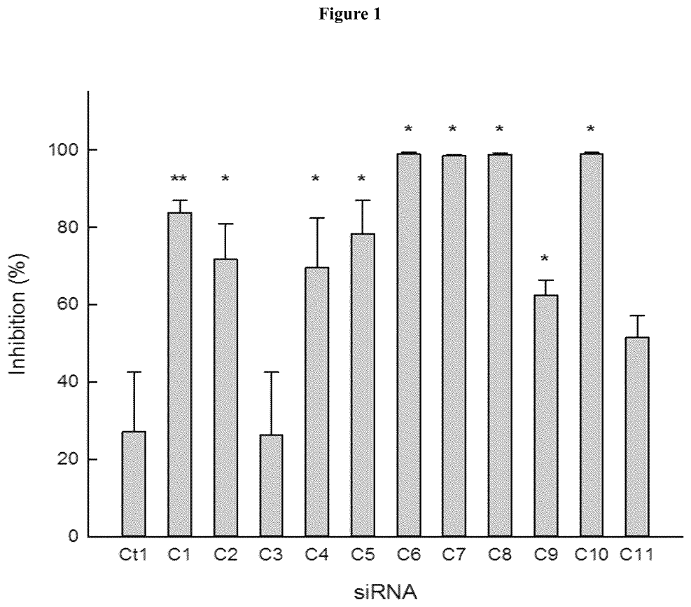

As shown in FIG. 1, all of the anti-SARS-CoV-2 siRNAs tested in this study showed certain level of inhibitory activity against SARS-CoV-2 genome RNA replication. Among the tested siRNAs, C6, C7, C8 and C10 showed greater than 98% inhibitory activity. FIG. 1.

Table 2 below summarizes the inhibitory activity of all tested exemplary siRNAs targeting SARS-CoV-2.

TABLE 2
Knockdown efficiency of SARS-CoV-2
RNA genome determined by qPCR.

| siRNA | Target position | Target gene* | SEQ ID NO | Inhibition (%) |
|---|---|---|---|---|
| C1 | 386-404 | Leader | 1 and 2 | 83.8 |
| C2 | 5208-5226 | PLP | 3 and 4 | 71.7 |
| C3 | 6740-6758 | PLP | 5 and 6 | 26.3 |
| C4 | 10119-10137 | 3CL | 7 and 8 | 69.6 |
| C5 | 10145-10163 | 3CL | 9 and 10 | 78.3 |
| C6 | 14666-14684 | POL | 11 and 12 | 98.8 |
| C7 | 15449-15467 | POL | 13 and 14 | 98.5 |
| C8 | 21586-21604 | Spike | 15 and 16 | 98.8 |
| C9 | 23451-23469 | Spike | 17 and 18 | 62.5 |
| C10 | 17461-17479 | Helicase | 19 and 20 | 99 |
| C11 | 26270-26288 | Envelope | 21 and 22 | 51.5 |

*The target position of each siRNA was listed according to the GenBank reference sequence NC_045512.

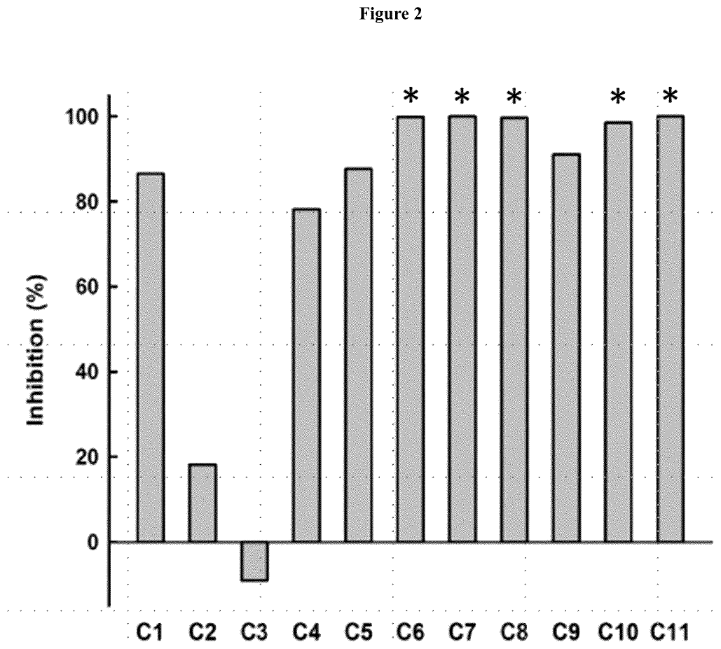

The virus titers in the culture medium were determined using the plaque assay described above. As shown in FIG. 2, most of the tested siRNAs showed inhibitory activity against SARS-CoV-2 production. Among them siRNAs C6, C7, C8, C10 and C11 showed greater than 98% inhibitory activity, consistent with the data received from the Vero cell assay.

In sum, the results obtained from this study demonstrate efficient inhibition of SARS-CoV-2 replication and production by siRNAs targeting SARS-CoV-2, including siRNA-C6, C7, C8, C10 and C11.

### Example 2: Development and Characterization of Modified siRNAs Targeting a Broad Range of SARS-CoV-2 Infections

The emergence of severe acute respiratory syndrome coronavirus variants has altered the trajectory of the COVID-19 pandemic and raised some uncertainty on long term efficiency of vaccine strategy. The development of new therapeutics against a wide range of SARS-CoV-2 variants is imperative. This study aims at developing and identifying potent siRNAs that are effective in inhibiting infections caused by a broad range of SARS-CoV-2 variants, including the currently dominant variants strains such as Alpha, Delta, Gamma and Epsilon. One modified siRNA, C6G25S, was identified as one example. It is reported here that C6G25S covers 99.8% of current SARS-CoV-2 variants and is capable of inhibiting the dominant strains, including those noted herein, with picomolar ranges of IC₅₀ in vitro. Moreover, C6G25S could completely inhibit the production of infectious virions in lungs by prophylactic treatment, and decrease 96.2% of virions by post-exposure treatment in K18-hACE2-transgenic mice, accompanying with a significant prevention of virus-associated extensive pulmonary alveolar damage, vascular thrombi, and immune cell infiltrations. The data suggests that the anti-SARS-CoV2 siRNAs disclosed herein, including C6G25S, would be effective therapeutic agents to combat the COVID-19 pandemic.

### Materials and Methods:

SARS-CoV-2-Specific siRNA Selection:

As of June 2020, there were 29,871 full-length SARS-CoV-2 genome sequences available from the Global Initiative on Sharing All Influenza Data (GISAID) website. These sequences were analyzed for 19-nucleotide stretches that showed at least 99% identity (high conservation) in the SARS-CoV-2 genome. Because RNA target accessibility affects the siRNA efficacy, the viral RNA secondary structure was evaluated based on RNA structure in vivo analyzed by Genome-wide dimethyl sulfate mutational profiling with sequencing (DMS-MaPseq) (Lan, et al., bioRxiv, 2020, doi: 2020.06.29.178343) and in silico prediction with RNAz (RNAz P<0.9) (Rangan, et al., 2020, RNA 26: 937-959). Sequences targeting viral regions with strong secondary structure were removed (RNAz P>0.9). A total of 674 siRNA candidates showed over 99% coverage and the targeted regions had low propensity for RNA secondary structure. We selected those candidates located within regions coding the viral leader, papain-like protease, 3C-like protease, RdRPs, helicase, spike protein, and the envelope protein for further off-target prediction and essential gene targeting. Off-target effects were predicted via blast with GRCh38 reference sequence database and candidates were filtered with the number of off-target gene 36. Off-target genes were further evaluated for their essential contribution to cell viability (Blomen et al., 2015, Science 350: 1092-6; Hart, et al., 2015, Cell 163: 1515-26; Wang, et al., 2015, Science 350: 1096-101). Candidates were selected with a low number of essential genes predicted to be targeted by the siRNA candidates (n 1). The top 11 siRNA candidates were identified for subsequent in vitro screening by viral RNA knockdown and plaque reduction assay in Vero E6 cells.

### Cells and Viruses:

Vero E6 cells were maintained in Dulbecco's modified Eagle's medium (DMEM) supplemented with 10% fetal bovine serum (FBS) at 37° C. with 5% CO₂. The human bronchial epithelial cell line BEAS-2B was maintained in RPI-1640 medium supplemented with 10% FBS. Sputum specimens obtained from patients infected with SARS-CoV-2 were maintained in viral transport medium. The virus in the specimens was propagated in Vero E6 cells in DMEM supplemented with 2 ug/mL tosylsulfonyl phenylalanyl chloromethyl ketone (TPCK)-trypsin (Sigma-Aldrich). Culture supernatant was harvested when cytopathic effect (CPE) was observed in more than 70% of cells, and virus titers were determined by plaque assay. The virus isolates used in in vitro siRNA screening and IC50 determination were hCoV-19/Taiwan/NTU13/2020 (A.3; EPI_ISL_422415), hCoV-19/Taiwan/NTU49/2020 (B.1.1.7; EPI_ISL_1010718), hCoV-19/Taiwan/NTU56/2021 (B.1.429; EPI_ISL_1020315), hCoV-19/Taiwan/CGMH-CGU-53/2021 (P.1; EPI_ISL_2249499), hCoV19/Taiwan/NTU92/2021 (B.1.617.2; EPI_ISL_3979387). The viruses used in the infection of K18-hACE2 transgenic mice were hCoV-19/Taiwan/4/2020 (B; EPI_ISL_411927) and hCoV-19/Taiwan/1144/2020 (B.1.617.2; not uploaded to GISAID database).

siRNA Screening in Vero E6 Cells:

Vero E6 cells were resuspended in culture medium at 2×10⁵ cells/mL and reverse-transfected with siRNA as follows: siRNA and Lipofectamine RNAiMAX (Thermo Fisher Scientific) were diluted with Opti-MEM I reduced serum medium (Thermo Fisher Scientific/Gibco) separately. The siRNA/Opti-MEM mixtures were added to the Lipofectamine RNAiMax/Opti-MEM mixtures. The siRNA-RNAiMAX mixtures (100 uL) were incubated for 10 min at room temperature. Vero E6 cells (500 ul, 2×10⁵ cells/mL) were then added to the siRNA-RNAiMAX mixtures and transferred into a 24-well plate.

After 24 h incubation, the siRNA-transfected Vero E6 cells were infected with SARS-CoV-2 virus at a multiplicity of infection (MOI) of 0.1. After 1 h incubation, the inoculum was removed and the cells were washed with phosphate-buffered saline (PBS). Fresh medium was added for incubation at 37° C. for 24 h. After that, culture supernatant was harvested for plaque assay (Cheng, et al., 2020, Cell Rep 33: 108254), and the total cellular RNA was extracted with a NucleoSpin RNA mini kit (Macherey-Nagel) to determine the amount of viral RNA by reverse transcription-quantitative polymerase chain reaction (RT-qPCR) of viral E gene on a QuantStudio 5 Real-Time PCR System (Applied Biosystems) using an iTaq Universal Probes One-Step RT-PCR Kit (Bio-Rad) (Cheng et al., 2020). The primers and probe targeting the SARS-CoV-2 were as follows: forward primer, 5′-ACA GGTACGTTAATAGTTAATAGCGT-3′ (SEQ ID NO: 62); reverse primer, 5′-ACATTGCAGCAGTACGCACACA-3′ (SEQ ID NO: 63); and probe, 5′-ACACTAGCCATCCTTACTGCGCTTCG-3′ (SEQ ID NO: 64).

Plasmid containing partial E fragment was used as a standard to calculate the viral load (copies/uL). All work involving SARS-CoV-2 virus was performed in the Biosafety Level-3 Laboratory of the First Core Laboratory, National Taiwan University College of Medicine.

siRNA Delivery Via Inhalation and Intranasal Instillation:

Inhalation delivery of siRNA was performed using a standard device consisting of a polycarbonate chamber connected to a Aeroneb Lab Nebulizer Unit at 0.4 mL/min. Mice (n=5/group) were placed into the chamber and aerosol was generated for 25 min from 10 mL normal saline containing 6 mg/mL siRNA for prophylactic treatment or 12 mg/mL siRNA for postexposure treatment. Mice were exposed to siRNA aerosol in the chamber for a total of 30 min. For intranasal administration, 50 ug siRNA in 50 uL of D5W was instilled into both nostrils (25 uL per nostril). To analyze the distribution and concentration of siRNA in the lungs and nasal cavity via inhalation, C57BL/6 mice were treated siRNA aerosol generated from 10 mL normal saline containing 6 mg/mL siRNA. The siRNA concentration of the aerosol in the chamber was quantified as follow. Aerosol samples were collected from the chamber using 0.5 mL syringes at 1, 2, 5, 15, and 25 min after aerosol was generated, and then passed through 100 uL nuclease-free water. The siRNA level in the nuclease-free water was subsequently determined by OD260. The maximum siRNA level, B, was calculated and resented as mg/L air aerosol.

Quantification of siRNA Level in Lungs and Nasal Mucosa:

C57BL/6 mice were sacrificed at different time-points after pulmonary delivery or intranasal instillation of siRNA. Livers and nasal mucosa were weighed and homogenized in 0.25% Triton X-100 in PBS to a final concentration of 100 mg/mL using a TissueLyser II (Qiagen) at 4° C. siRNA level was quantified by stem-loop RT-qPCR (Brown, et al., 2020, Nucleic Acids Res 48: 11827-11844). Briefly, homogenized samples were heated to 95° C. for 10 min, briefly vortex, and cooled on ice for 10 min. The resultant tissue lysate was collected after centrifugation at 20,000×g for 20 min at 4° C. Antisense-specific cDNA was generated from tissue lysate using a stem-loop cDNA primer:

(SEQ ID NO: 65)
5′-GTCGTATCCAGTGCAGGGTCCGAGGT
ATTCGCACTGGATACGACAACTGTCA-3′.

qPCR was performed on a QuantStudio 6 Flex Real-Time PCR System (Thermo Fisher Scientific) using Power SYBR Green PCR Master Mix (Thermo Fisher Scientific).

Forward primer,
(SEQ ID NO: 66)
5′-AAGCGCCTAAATTACCGGGTT-3′;
Reverse primer,
(SEQ ID NO: 67)
5′-GTGCAGGGTCCGAGGT-3′

Antisense strand level was quantified using a standard curve generated by spiking the synthetic siRNA into the corresponding naïve tissue matrix of the same concentration.
siRNA Treatment and Virus Infection of K18-hACE2 Mice:

Eight- to 16-week-old male and female K18-hACE2 transgenic mice (McCray, et al., 2007, J Virol 81: 813-21) were purchased from The Jackson Laboratory and inbred in Laboratory Animal Center of National Taiwan University College of Medicine (Taipei, Taiwan, ROC). For the prophylactic treatment, mice were treated with aerosolized siRNA and the subsequent intranasal instillation of siRNA daily for 3 days before virus infection (D-1 to D-3). Twenty-four hours after the last siRNA treatment (DO), mice were anesthetized with Zoletil/Dexdomitor and infected intranasally with 104 plaque-forming units (pfu) of SARS-CoV-2 in 20 uL of DMEM, followed by Antisedan administration. For postexposure treatment, mice were treated with aerosolized siRNA at DO and 1-day post infection. Two days post infection, infected mice were sacrificed to collect lungs. All work with SARS-CoV-2 was conducted in a Biosafety Level (BSL)-3 and BSL-4 Laboratories at Institute of Preventive Medicine, the National Defense Medical Center (Taiwan, ROC).

### Quantification of SARS-CoV-2 RNA and Infectious Virus in Lungs:

Lungs were suspended in 1 mL DMEM supplemented with 1× antibiotic-antimycotic (Gibco) before further homogenization using beads in a Precellys tissue homogenizer (Bertin Technologies). Tissue homogenates were clarified by centrifugation at 12,000×g for 5 min at 4° C. The supernatants were collected for determination of infectious virus by plaque assay and viral RNA titers by RT-qPCR. The clarified lung homogenates were mixed with a fivefold excess of TRI reagent (Sigma-Aldrich). RNA was extracted following the manufacturer's instructions (TRI reagent). The extracted RNA was dissolved in 100 uL nuclease-free water. Viral RNA was quantified using SensiFAST Probe No-ROX One-Step Kit (catalog No. BIO-76005, Bioline) on the LightCycler 480 (Roche Diagnostics). Primers and Probe targeting the viral E gene were purchased from Integrated DNA Technologies (catalog Nos. 10006888, 10006890, 10006893). RT-qPCR was performed with 500 ng of total RNA, 400 nM of each forward and reverse primer, and 200 nM probe in a total volume of 20 uL. The cycling conditions were as follows: 55° C. for 10 min, 94° C. for 3 min, and 45 cycles of 94° C. for 15 s and 58° C. for 30 s. The amount of viral RNA was calculated using a standard curve constructed from an RNA standard. The virus titer in the clarified lung homogenates was quantified using a plaque assay. Briefly, Vero E6 cells (1.5×10⁵ cells/well) were seeded in 24-well tissue culture plates in DMEM supplemented with 10% fetal bovine serum (FBS) and antibiotics. The 10-fold serial diluted homogenates were inoculated into Vero E6 cells for 1 h at 37° C. with shaking occasionally. After removing the supernatant, cells were washed once with PBS, overlaid with 1.55% methylcellulose in DMEM with 2% FBS, and then incubated for another 5 days. The methylcellulose overlays were removed after 5 days of incubation. Cells were fixed with 10% formaldehyde for 1 hr, and stained with 0.5% crystal violet. Plaques were counted to calculate PFU/g according to lung weight.

### In Situ Hybridization:

Lungs and nasal cavities were fixed in formalin, embedded in paraffin, and sectioned at 4 um thickness. The localization of C6 siRNA was investigated in tissue sections using the miRNAscope Intro Pack HD Reagent Kit RED-Mmu (Advanced Cell Diagnostics [ACD]) according to ACD's formalin-fixed paraffin-embedded tissue protocol. The probe for the detection of C6 siRNA was custom-synthesized by ACD. The hybridization signal of C6 siRNA was visualized by Fast Red, followed by counterstaining with hematoxylin. SARS-CoV-2 RNA was detected using the RNAscope 2.5 HD Reagent Kit-Brown (ACD) and RNAscope Probe-V-nCoV-2019-S(ACD). The hybridization signal of SARS-CoV-2 RNA was visualized using a 3,3′-diaminobenzidine (DAB) reagent. RNA quality in the tissue sections was verified using the probe targeting U6 snRNA as a positive control and scrambled probe as a negative control. The whole-slide images were acquired using a Ventana DP200 slide scanner (Roche Diagnostics) and processed using HALO software (Indica Labs). Quantitative comparison of ISH signals was analyzed using the HALO software with RNAscope modules.

### Immunohistochemical Analysis:

Formalin-fixed, paraffin-embedded lung sections were dewaxed and rehydrated and antigen retrieval was performed with Tris-EDTA buffer (pH 9.0). Endogenous peroxidase in the sections was quenched with 3% hydrogen peroxide and the tissue immunostained using a Histofine Mousestain Kit (Nichirei Biosciences) according to the manufacturer's protocol. SARS-CoV-2 spike protein, neutrophils, macrophages and CD8⁺ T cells were detected by incubation with anti-SARS-CoV/SARS-CoV-2 (COVID-19) spike antibody (Clone 1A9, Genetex), anti-LY-6G (Clone 1A8, BD), anti-F4/80 (Clone Rb167B3, Synaptic Systems) and anti-CD8 (Clone, Synaptic Systems) in primary antibody diluent (ScyTek) at 4° C. overnight. Sections were stained using DAB reagent, counterstained with hematoxylin, and then dehydrated and mounted under cover slips. Whole slides were scanned on a Ventana DP200 slide scanner (Roche Diagnostics) and analyzed using HALO software (Indica Labs). The severity of lung injury was assessed based on the presence of neutrophils in the alveolar space, neutrophils in the interstitial space, hyaline membranes, proteinaceous debris is filling the airspaces, and alveolar septal thickening, following the method as described (Matute-Bello et al., 2011, Am J Respir Cell Mol Biol 44: 725-38).

### In Vitro Evaluation of Cytokine Release by Human PBMC:

The peripheral blood mononuclear cells (PBMC) from healthy donors were obtained from StemExpress with Institutional Review Board (IRB) approval (IRB No. 20152869). PBMCs were resuspended in RPMI 1640 medium supplemented with 10% FBS. A total of 1×10⁵ viable PBMC were added to each well of a 96-well culture plate. After 4 h, cells were treated with different concentrations (40, 20, 10, and 5 uM) of siRNA, 800 ug/mL CpG, and 100 ug/mL poly(I:C) for 40 h. Concentrations of cytokine IL-1alpha, IL-1beta, IL-6, IL-10, TNF-alpha and IFN-gamma in the supernatant were quantified using the Cytometric Bead Assay (CBA) Flex Set (BD Biosciences) according to the manufacturer's instructions. The data were collected on a FACS LSRFortessa flow cytometer (BD Biosciences) and analyzed using FCAP Array Software (version 3.0, BD Biosciences).

### Genome-Wide Off-Target Analysis Using RNA-Seq:

Beas-2B cells were seeded at 5×10⁵ cell/well into 6-well culture plates and incubated for 18 h. siRNA (10 nM) was then transfected into Beas-2B cells using Lipofectamine RNAiMAX (9 ul/well, Thermo Fisher Scientific) following the manufacturer's protocol. After 24-h transfection, cells were washed twice with 1× Dulbecco's PBS and solubilized in TRIzol reagent (Thermo Fisher Scientific). Total RNA was extracted following the manufacturer's instructions and treated with DNase to avoid genomic DNA contamination. Purity (A260/A280 and A260/A230 ratios) and quality (RIN≥8.0) of the extracted RNA were determined using a NanoDrop 2000 spectrophotometer (Thermo Scientific) and an Agilent 2100 bioanalyzer (Agilent Technologies, Santa Clara, CA). Quality of all extracted RNA samples was A260/A280≥1.9, A260/A230≥2, and RIN=10.0. RNA-seq Libraries was prepared using TruSeq Stranded Total RNA Library Prep Gold (Illumina) and sequenced on the NovaSeq 6000 sequencer (Illumina) according to the manufacturer's instructions. Average of 86.7 million reads per sample was obtained from 2×150-bp paired-end sequencing. Raw RNA reads were filtered with minimal mean quality scores of 20 using SeqPrep and Sickle. Filtered reads were aligned to the human genome (GRCh.38.p13) using HISAT2 and then assembled using StringTie. The gene expression level was qualified by RSEM and normalized by TPM (transcripts per million). Differentially expressed genes were identified as those with at least threefold difference between siRNA-treated and no siRNA-treated groups at the Benjamini-Hochberg false discovery rate adjusted for P 0.001. Off-target gene profile was evaluated from the number and possible cellular impact of down-regulated genes. The expression level of down-regulated genes was further confirmed by RT-qPCR. First-strand cDNA was synthesized with Maxima First-Strand cDNA Synthesis kit (Thermo Fisher Scientific) and 2 ug of total cellular RNA. qPCR was carried out on a LightCycler 480 (Roche Diagnostics) using SYBR Green I Master (Roche Diagnostics). Each sample was assayed in triplicate to determine an average threshold cycle (Ct) value. Gene expression fold change was calculated using the ΔΔCt method. The mRNA level of each gene was normalized to constitutively expressed GAPDH mRNA.

### Acute and Repeated-Dose Toxicity Studies:

To assess the acute toxicity of C6G25S, male Sprague Dawley rats were obtained from BioLASCO (Taipei, Taiwan, ROC). C6G25S in D5W was administered to 7-week-old rats (3 per group) via intranasal instillation at 20, 40, or 75 mg/kg at a dose volume of 0.42 ml/kg. The rats were then observed for 7 days. Repeated-dose toxicity was conducted on male Bltw:CD1(ICR) mice obtained from BioLASCO. C6G25S (2, 10, or 50 mg/kg) was intranasally instilled (1.67 mL/kg) to 8-week-old mice (3 per group) daily for 14 days. During the study, the body weight, food consumption, and general status of the animals were monitored. At the end of each study, organs and peripheral blood were collected. Acute toxicity in rats was assessed based on the clinical signs, body weight and food consumption, hematology, blood biochemistry, and microscopic pathology of the nasal cavity and lung. The assessment of repeated-dose toxicity also included the microscopic pathology of the heart, liver, spleen, and kidney.

### CCK-8 Cytotoxicity Assay:

Beas-2B cells were seeded at 1.77×10⁴ cell/well into 96-well culture plates and incubated for 18 h. Cells were then treated with various concentrations of C6G25S (40, 20, 10, 5, and 0 uM) in triplicate for 24 h. CCK-8 solution (10 uL) was added to each well, and cells were incubated for another 3 h. Medium only with CCK-8 solution and medium without C6G25S served as blank and normal control, respectively. The absorbance at 450 nm was measured with a Multiskan Sky Microplate Spectrophotometer (Thermo Fisher Scientific). The relative cell viability/cytotoxicity was calculated according to the manufacturer's instructions.

### Results

I. Selection and Screening of Highly Specific and Potent siRNA Against SARS-CoV-2

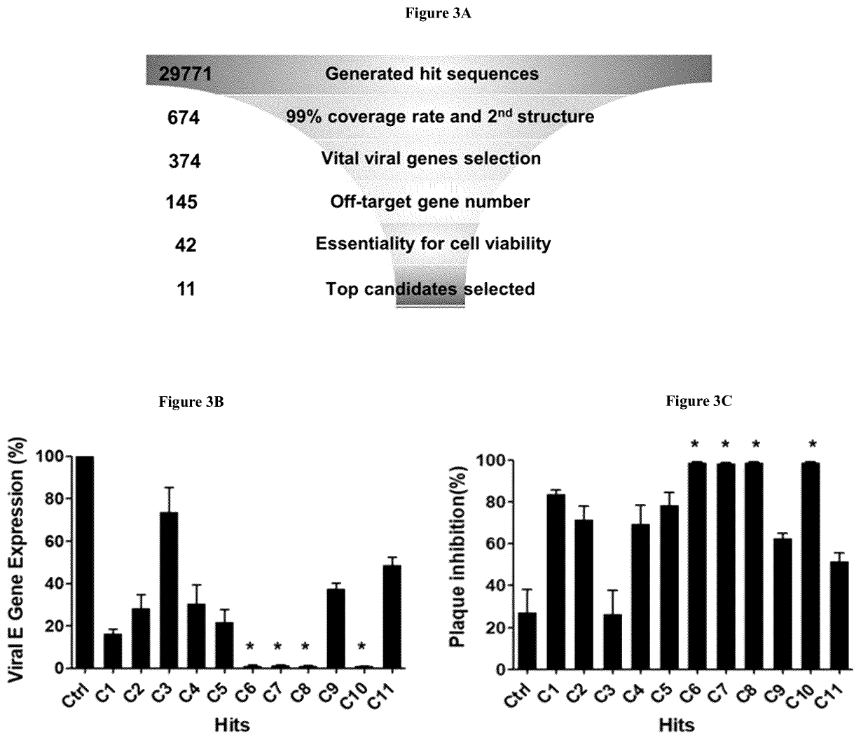

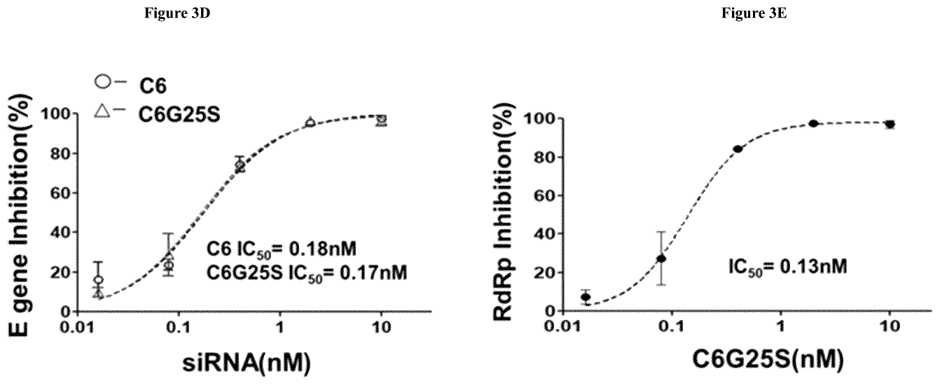

Coronavirus has the largest genome of all known RNA viruses, ranging from 26 to 32 kb (Woo, et al., 2010, Viruses 2: 1804-20). To identify a highly potent and specific siRNA sequence against SARS-CoV-2 variants, a systematic and comprehensive selection strategy was applied. As shown in FIG. 3A, the filtering process began with a segmentation of the virus genome into 29,771 hit sequences of 19-nucleotide stretches. Next, 674 siRNA candidates with over 99.8% coverage rate among 29,871 SARS-CoV-2 genomes and their corresponding targeting regions with low propensity for secondary structure were selected (Lan, et al., bioRxiv, 2020, doi: 2020.06.29.178343; Rangan, et al., 2020, RNA 26: 937-959). Further evaluating the location of the siRNA binding sites on the vital genes involved in virus replication and infection, 374 located in regions encoding the viral leader, papain-like protease, 3C-like protease, RNA dependent RNA polymerase (RdRp), helicase, spike protein, and the envelope protein were isolated. After removing those with high potential off-target effects to human transcriptome and targeting genes essential for cell viability, the top 11 siRNA with the lowest predicted off-target effects and high predicted efficacy were selected and the detail sequences with key comparison information were shown in Table 3 below. The effectiveness of selected siRNA to protect Vero E6 cells against SARS-CoV-2 infection was verified. In vitro screening in Vero E6 cells showed that C6, C7, C8, and C10 were capable of inhibiting up to 99.9% of both viral envelope gene expression and plaque-forming virions production at a concentration of 10 nM (FIGS. 3B-3C and Table 3).

TABLE 3
siRNA Candidates Against SARS-CoV-2.

| | | SARS-CoV2 | 2^{nd} | Numbers of |
|---|---|---|---|---|
| siRNA | Target | strains | structure | predicted |
| Candidate | genes | Coverage Rate | prediction | off-target genes |
| C1 | Leader | 99.60% | Weak | 36 |
| C2 | PLP | 99.30% | None | 15 |
| C3 | PLP | 99.60% | Weak | 11 |
| C4 | CLPro | 99.80% | Weak | 20 |
| C5 | CLPro | 99.70% | None | 3 |
| C6 | RDRP | 99.80% | None | 9 |
| C7 | RDRP | 99.90% | Weak | 23 |
| C8 | Spike | 99.20% | None | 16 |
| C9 | Spike | 99.90% | Weak | 13 |
| C10 | Helicase | 99.70% | None | 14 |
| C11 | Envelope | 99.90% | Weak | 12 |

The start and end sites of 11 siRNA candidates listed in Table 3 and the located genes directly targeted by siRNA candidates are based on the reference genome of SARS-CoV-2, NC_045512.2. Coverage rates were calculated by using the 29,871 full genome SARS-CoV-2 sequences from the Global Initiative on Sharing All Influenza Data (GISAID) website. For the secondary structure prediction, the target site confirmed as a non-structured area was labeled as none (Lan, et al., bioRxiv, 2020, doi: 2020.06.29.178343; Rangan, et al., 2020, RNA 26: 937-959). Those site with an RNAz P<0.9 were predicted to have propensity to form secondary structures and labeled as weak. Candidates selected for high anti-SARS-CoV2 efficacy were labeled in bold.

The target sites of C6, C7, C8, and C10 on viral genome were listed in Table 3 above. C6, C8, and C10 were then fully modified into C6G25S, C8G25S and C10G31A by 2′-O-methyl, 2′-fluoro, and phosphorothioate (PS) substitution for nuclease protection (Hu, et al., 2020, Signal Transduct Target Ther 5: 101) as shown in Table 4 below.

TABLE 4
Structures of Exemplary Modified siRNAs

| | | | SEQ |
|---|---|---|---|
| | | | ID |
| SiRNA | Strand | Sequence (5′ --3′) | no. |
| C6G25S | Sense | mC*mU*mGmUfCmAfAfAfCf | 68 |
| | | CmCmGmGmUmAmAmU*mU*mU | |
| | Antisense | mA*fA*mAfUfUmAmCfCfGmGm | 69 |
| | | GmUmUfUmGfAmCfAmG*mU*mU | |
| C8G25S | Sense | mG*mC*mCmAfCmUfAfGfUf | 70 |
| | | CmUmCmUmAmGmUmC*mA*mA | |
| | Antisense | mU*fU*mGfAfCmUmAfGfAmGm | 71 |
| | | AmCmUfAmGfUmGfGmC*mU*mU | |
| C10G31A | Sense | CGCmA*fC*mAfUmUfGmCfUmA | 72 |
| | | mAmCmUmAmA*mG*mA | |
| | Antisense | mU*fC*mUfUmAfGmUfUmAfGm | 73 |
| | | CfAmAfUmGfUmGmCmG*mU*mU | |

m: 2′-O-methyl
f: 2′-fluro
*PS bond

Half-maximal inhibitory concentration (IC₅₀) for C6G25S, C8G25S and C10G31 were determined as 0.17, 1.25 and 0.94 nM, respectively. C6G25S was selected for subsequent in vitro and in vivo experiments for its lowest IC50 value and numbers of off-target genes predicted in silico.

Whole transcriptome analysis using next generation sequencing (NGS) showed that the modification of C6 reduced the total number of off-target genes in BEAS-2B cells from 21 to 15. The 15 off-target genes were further verified by RT-qPCR and only four genes, including CXCL5, REEP3, SGPP1, and ARTN were confirmed to be true off-target genes (Table 4). None are known to be essential for cell survival, demonstrating that C6G25S is a safe and highly specific siRNA candidate. Furthermore, the major off-target gene of C6G25S, CXCL5, is a chemotactic factor secreted by lung epithelial cells and has a participatory role in COVID-19-associated pathogenesis by induction of neutrophil infiltration and acute lung injury (Nouailles, et al., 2014, J Clin Invest 124: 1268-82), and has a participatory role in COVID-19-associated pathogenesis (Tomar, et al., 2020, Cells 9). This data suggest that C6G25S might have a unique dual effect that can simultaneously inhibit SARS-CoV-2 infection and reduce the risk of severe illness.

Moreover, C6G25S and unmodified C6 were found to have a similar IC₅₀ for inhibiting the viral envelope gene (0.17 and 0.18 nM, respectively) (FIG. 3D). The expression of RdRp, the direct target for C6G25S, was also analyzed and revealed an IC50 of 0.13 nM (FIG. 3E). These data suggest that C6G25S was a high potent siRNA with a picomolar range of IC₅₀ in the inhibition of SARS-CoV-2 and the low off-target property implies that C6G25S might have a better safety profile beneficial for therapeutic use.

TABLE 5
Genome-wide off-target evaluation via RNA-seq and subsequent RT-qPCR confirmation

| | | | | | Knockdown |
|---|---|---|---|---|---|
| | | TPM | TPM | Inhibition % | efficacy % |
| Gene Name | Description | (Control) | (C6G25S) | (NGS) | (qPCR) |
| STON1-GTF2A1L | Function unknown | 0.26 | 0.08 | 69 | 0 |
| ARTN | Artemin | 1.55 | 0.36 | 77 | 46 |
| SGPP1 | Sphingosine-1-phosphate | 7.06 | 2.01 | 72 | 81 |
| | phosphatase 1 | | | | |
| JUND | AP-1 transcription factor subunit | 7.94 | 1.58 | 80 | 0 |
| TCHH | Trichohyalin: Intermediate | 0.18 | 0.06 | 67 | 0 |
| | filament-associated protein | | | | |
| CXCL5 | C-X-C motif chemokine ligand 5 | 0.19 | 0.005 | 97 | 91 |
| NPIPB6 | Nuclear Pore Complex | 0.3 | 0.006 | 98 | 11 |
| | Interacting Protein Family | | | | |
| | Member B6 | | | | |
| FAM187A | Family With Sequence Similarity | 0.53 | 0.15 | 72 | 0 |
| | 187 Member A | | | | |
| NPIPA3 | Nuclear Pore Complex | 1.3 | 0.12 | 91 | 0 |
| | Interacting Protein Family | | | | |
| | Member A3 | | | | |
| CDRT4 | CMT1A Duplicated Region | 0.91 | 0.35 | 62 | 0 |
| | Transcript 4 | | | | |
| TNFSF12-TNFSF13 | TNFSF12-TNFSF13 | 0.54 | 0.007 | 99 | 0 |
| | Readthrough | | | | |
| | (TNF family) | | | | |
| CTAGE15 | CTAGE family member 15 | 0.17 | 0.005 | 97 | 0 |
| SMIM11B | Small integral membrane protein | 10.08 | 1.01 | 90 | 9 |
| | 11B: Function unknown | | | | |
| TBC1D3 | TBC1 domain family member 3 | 0.36 | 0.006 | 98 | 0 |
| TBCEL-TECTA | TBCEL-TECTA Readthrough | 0.53 | 0.01 | 98 | 17 |
| REEP3 | Receptor Accessory Protein 3 | 22.81 | 7.74 | 66 | 74 |

Table 5 shows the down-regulated genes with fold change 3 in C6G25S-treated Beas-2B cells (10 nM C6G25S) compared with no siRNA control and presented with inhibition % based on the expression level, transcripts per millions (TPM) of RNA-seq. The mRNA level was confirmed by RT-qPCR and normalized to the GAPDH reference gene.

### II. Inhibition of Multiple Strains of SARS-CoV-2 In Vitro by C6G25S

According to the World Health Organization's website as of Aug. 17, 2021, Alpha (B.1.1.7), Beta (B.1.351), Gamma (P.1), and Delta (B.1.617.2) are recognized as SARS-CoV-2 variants of concern (VOC), and Eta (B.1.525), Iota (B.1.526), Kappa (B.1.617.1), and Lambda (C.37) are recognized as SARS-CoV-2 variants of interest (VOI).

The data presented in FIGS. 4A-4B shows that C6G25S can tackle multiple variants including Alpha, Gamma, Delta, and Epsilon, with picomolar range of IC₅₀. Moreover, the C to U transversion of some Alpha variants located at the 9^{th} nucleotide in C6G25S targeting site is tolerated by siRNA recognition (Huang, et al., 2009, Nucleic Acids Res 37: 7560-9) and can still be inhibited by C6G25S with a IC₅₀ of 0.46 nM (FIG. 4B, left top).

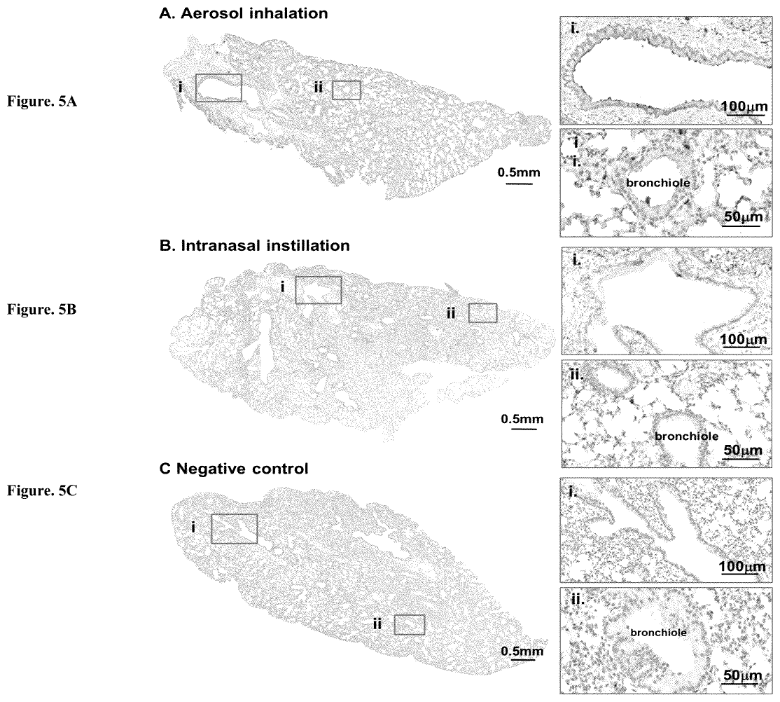

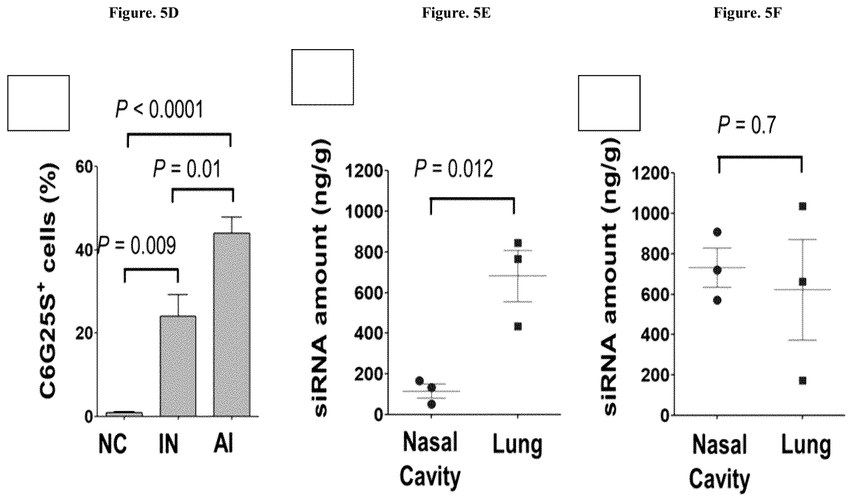

The C6 candidate was designed to target a highly conserved region with no mutations from SARS-CoV-1 to SARS-CoV-2. The upper part of FIG. 5A presents the location of C6, and the genetic map of the VOC, VOI, and other strains listed as indicated above. The lower part of FIG. 4A shows the sequence alignment of C6 and RdRp, which is located in the front section of ORF1b. As observed in FIG. 4B, C6G25S is capable of inhibiting significantly a variety of SARS-CoV-2 variants, with IC₅₀ of 0.46 nM for the Alpha variant, 0.5 nM for Gamma, 0.09 nM for Delta, and 0.73 nM for Epsilon variant. These data demonstrate that C6G25S can target highly conserved region of viral RdRp gene and is a highly effective therapeutic agent to suppress multiple strains of SARS-CoV-2.

### III. In Vivo Evaluation of Pulmonary Administration Route of C6G25S.

Direct delivery of C6G25S via intranasal instillation (IN) or aerosol inhalation (A1) were next implemented as considering that the siRNA carriers, such as virus-like particles, lipid nanoparticles and cell penetrating peptides, could cause adverse immune stimulation or cytotoxicity (Farkhani, et al., Nanobiotechnol 10: 87-95; Slutter, et al., 2011, J Control Release 154: 123-30; Vangasseri, et al., 2006, Mol Membr Biol 23: 385-95; Wilson, et al., 2009, Mol Genet Metab 96: 151-7). Furthermore, naked siRNA delivery via IN or A1 has been widely applied to knockdown specific gene or inhibit viral infection in lungs of different animal species (Bitzo, et al., 2005, Nat Med 11: 50-5; Kandil, et al., 2019, Ther Deliv 10: 203-206; Zafra, et al., 2014, PLoS One 9: e91996), including nonhuman primates (Li, et al., 2005, Nat Med 11: 944-51) and humans (DeVincenzo, et al., 2010, Proc Natl Acad Sci USA 107: 8800-5; Gottlieb, et al., 2016, J Heart Lung Transplant 35: 213-21).

To assess whether IN or AI can provide even distribution of C6G25S to the lungs, mice exposed to C6G25S by either IN or AI were sacrificed humanely and their lungs were collected for in situ hybridization (ISH) with a C6G25S-specific probe. The hybridization signal showed that C6G25S was evenly distributed throughout the bronchi, bronchioles, and alveoli of mice in the AI group (FIG. 5A), whereas uneven distribution was observed in the lungs of mice in the IN group (FIG. 5B). Lung from mice without C6G25S treatment served as a negative control (FIG. 5C). Moreover, there were twice as many C6G25S probe-stained positive cells in the AI group compared with that of the IN group (FIG. 5D). These data indicated that aerosol inhalation can distribute C6G25S more evenly and efficiently throughout the whole lungs than intranasal instillation.

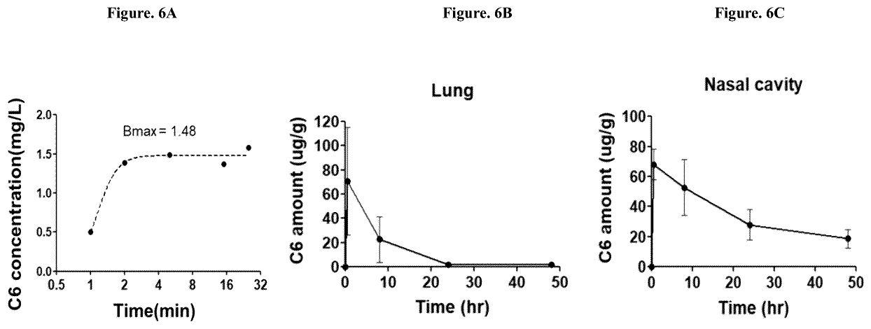

Air samples were collected from the inhalation chamber at various time points during aerosol generation to calculate the dose of C6G25S deposited by AI, and the C6G25S concentration. The C6G25S concentration in the chamber reached a maximum within 2 min and was maintained at 1.48 mg/L (FIG. 6A). To determine the deposition of C6G25S delivered by IN and AI, nasal cavity and whole lungs from C6G25S treated mice were collected to quantify the distribution of C6G25S by stem-loop reverse transcription-polymerase chain reaction (RT-PCR). The C6G25S concentrations in the lung were 5.8 times higher than that in the nasal cavities when C6G25S was delivered by AI (FIG. 5E). By contrast, similar concentrations were detected in the nasal cavities and lungs when C6G25S was delivered by IN, despite, a significant variation of siRNA level in lungs was observed (FIG. 5F). The elimination rate of C6G25S in lungs and nasal cavities was quantified at different time points for mice after AI and IN treatment, and a rapid decrease of C6G25S in both nasal cavity and lung tissues was observed within 24 hrs (FIG. 6B and FIG. 6C). These findings suggest a combination of IN and AI might have advantage to achieve thorough and stable prophylactic protection.

### IV. Prophylactic and Post-Exposure Treatment of C6G25S.

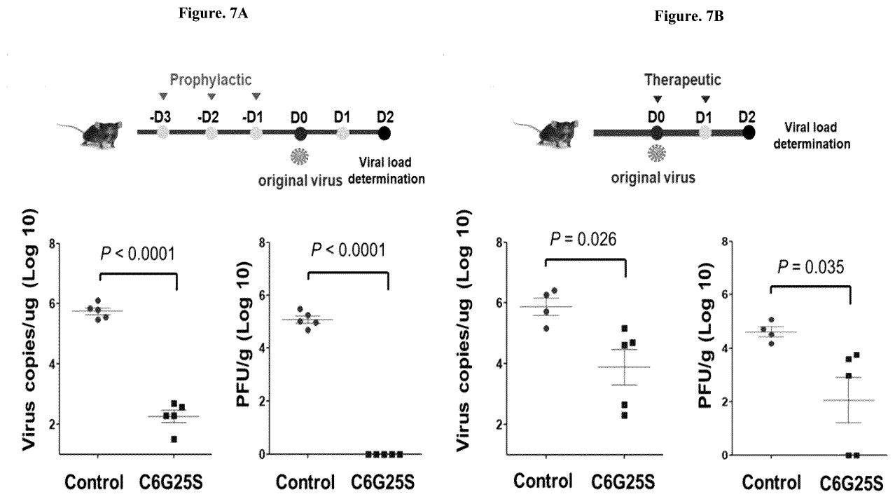

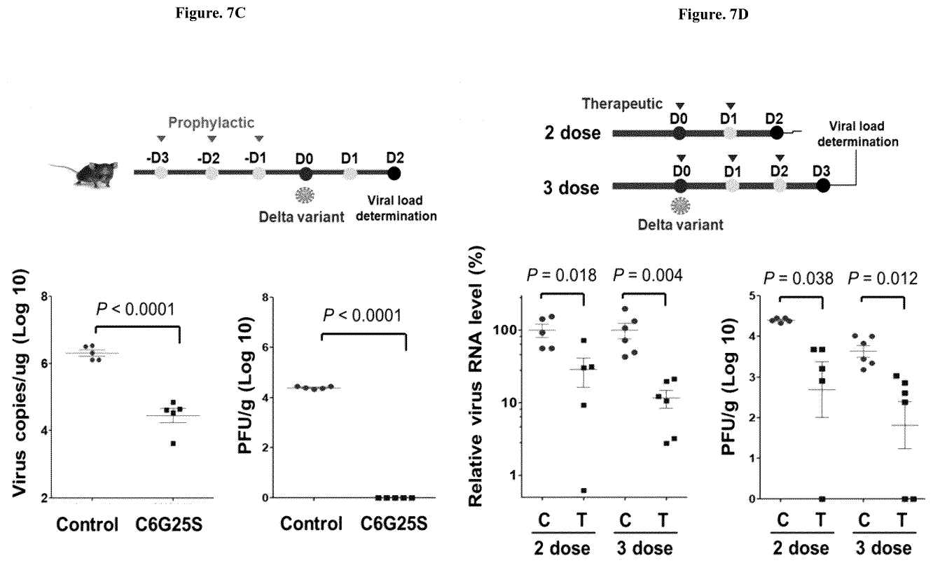

To determine whether C6G25S is protective in vivo, K18-hACE2 transgenic mice receiving a prophylactic or postexposure administration of C6G25S were used as an animal model. Viral quantification at two days post-infection (dpi) based on previous study (Winkler, et al., 2020, Nat Immunol 21: 1327-1335) was first evaluated. Viral RNA copies were reduced by 99.95% in the prophylactic group (FIG. 7A, left panel) and by 96.2% in the postexposure group (FIG. 7B, left panel). No plaque-forming virions were detected in the prophylactic group (FIG. 7A, right panel) and a significant decrease of infectious virions by 96% was observed in the postexposure group (FIG. 7B, right panel). Considering that the SARS-CoV-2 Delta variant is globally pervasive and responsible for vaccine breakthrough cases, the therapeutic effects of C6G25S on this particular variant in K18-hACE2 transgenic mice were explored in this example. Prophylactic treatment of the infected mice with C6G25S resulted in a 98.3% reduction of viral RNA (FIG. 7C, left panel) with no detectable infectious virions (FIG. 7C, right panel) in the lungs as compared with that of the control group. Two post-exposure groups, including two doses and three doses of C6G25S treatment after Delta variants infection, were tested. A significant inhibition of viral RNA by 72% and 88% was observed for two dose and the three dose groups, respectively (FIG. 7D, left panel). Similar reduction of infectious virions was also noted, 90.5% for the two dose and 92.7% for the three dose groups (FIG. 7D, right panel). This data demonstrates that pulmonary delivery of C6G25S possesses a strong antiviral activity in vivo against SARS-CoV-2 and its Delta variants in both prophylactic and post-exposure treatment.

### V. Inhibition of Spike Protein Expression by C6G25S.

Lungs from the infected K18-hACE2 transgenic mice without C6G25S treatment were collected and sectioned on a microtome. Immunohistochemistry demonstrated overexpression of spike proteins throughout bronchi, bronchioles, and alveoli. Moreover, pathological features of COVID-19 were observed, including pneumocyte proliferation, loss of empty space in alveoli (Wang, et al., 2020a, EBioMedicine 57: 102833), formation of syncytial multinucleated cells (Bussani, et al., 2020, EBioMedicine 61: 103104), and thrombosis (Bussani, etal., 2020, EBioMedicine 61: 103104). By contrast, lung tissue from mice with prophylactic C6G25S treatment showed a significant reduction of spike protein expression and COVID-19-associated pathological features.

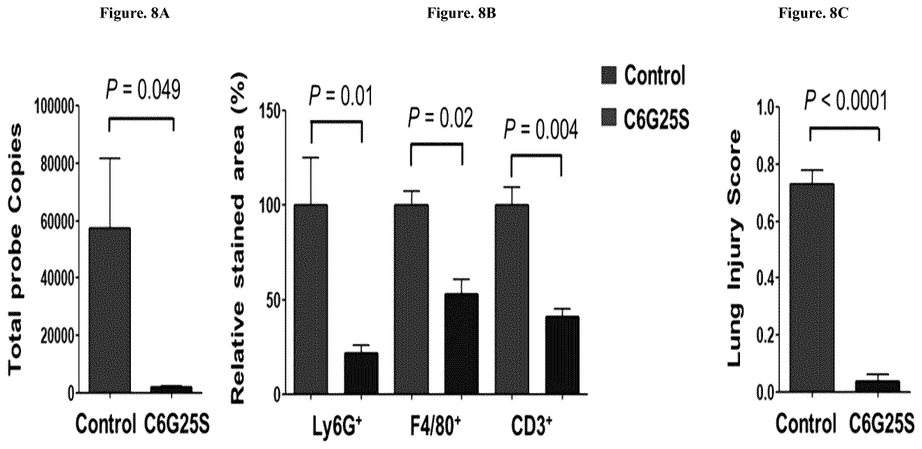

Furthermore, a significant decrease of viral RNA by ISH was also observed after C6G25S prophylactic treatment with the respective viral RNA signal quantified and shown in FIG. 8A. To determine whether the SARS-CoV-2 induced infiltration of neutrophil (Wang, et al., 2020b, Front Immunol 11: 2063), lymphocyte (Puzyrenko, et al., 2021, Pathol Res Pract 220: 153380), and macrophage (Wang, et al., 2020a, EBioMedicine 57: 102833) and acute lung inflammation could be alleviated by C6G25S treatment, lung tissue from untreated and treated mice was further stained using anti-Ly6G (neutrophil), anti-F4/80 (macrophage), and anti-CD3 (lymphocyte). Infiltration of neutrophils, macrophages, and lymphocytes was observed in the lungs of infected mice, but a significant decrease of immune cell infiltration was observed upon C6G25S treatment. The ratio of the infiltrated immune cell area to the whole section area was determined and normalized to the control group. The positively stained area of neutrophil, macrophage and CD3⁺ lymphocytes area reduced 78.2%, 46.9% and 62.4% by C6G25S treatment, respectively (FIG. 8B). The lung injury was evaluated using the scoring system published by the American Thoracic Society in 2011 (Matute-Bello, et al., 2011, Am J Respir Cell Mol Biol 44: 725-38). C6G25S treatment significantly reduced SARS-CoV-2-associated lung injury in the K18-hACE2 transgenic mice (FIG. 8C).

### VI. Non-Immunogenicity of C6G25S.

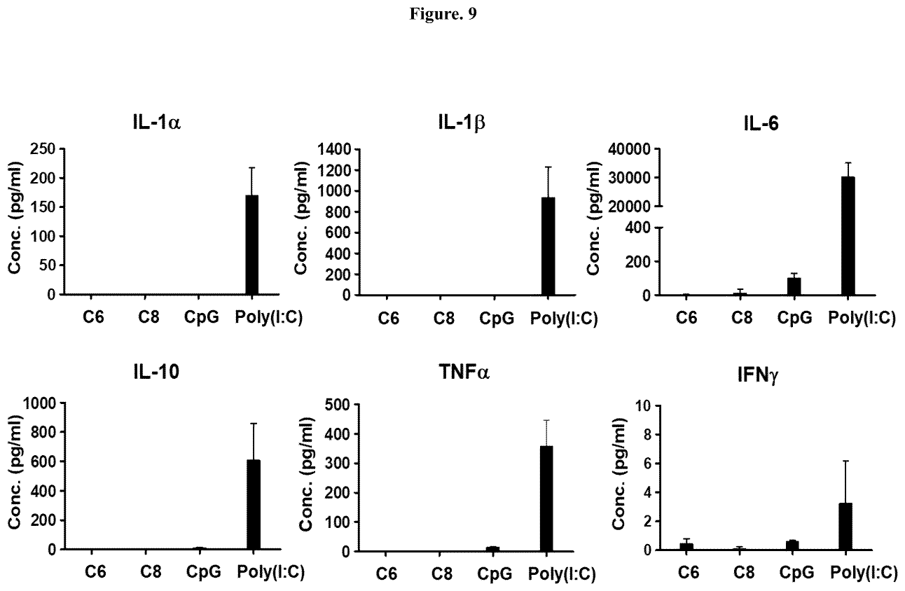

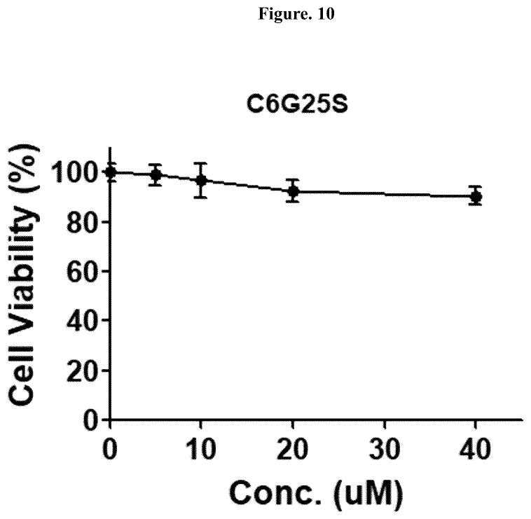

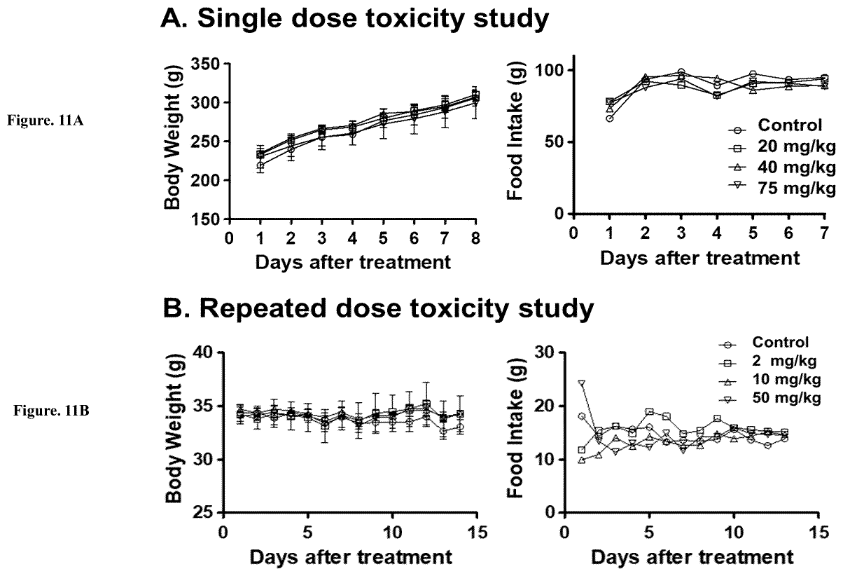

To determine the clinical utility of C6G25S, human peripheral blood mononuclear cells were cocultured with 10 uM of C6G25S, and no cytokines, such as interleukin (IL)-1alpha, IL-1beta, IL-6, IL-10, tumor necrosis factor-alpha, nor interferon-gamma were significantly induced (FIG. 9). Additionally, BEAS-2B, a human cell line from normal bronchial epithelium was exposed to a higher concentration of C6G25S in a cytotoxicity assay and no cytokine induction was observed (FIG. 10). The results show that C6G25S is not immunogenic to human immune cells.

### VII. Single-Dose and Multiple-Dose Toxicity Studies

To determine the potential adverse effects of C6G25S in vivo, a single-dose toxicity study was conducted in Sprague Dawley rats with a single dose up to 75 mg/kg, and a 14-day repeated-dose study was conducted in mice with daily dose up to 50 mg/kg.

In both studies, no animal death, body weight change, or drug-related adverse effect was observed within the monitoring period. See FIG. 11A for single dose toxicity study and FIG. 11B for repeated dose toxicity study.

The histopathology, hematology and blood biochemical analysis revealed no abnormalities in a single-dose toxicity study. The results are provided in Tables 6-8 below. See also FIG. 11A.

TABLE 6
Histopathology of Nasal Cavity and Lung in Single-dose Toxicology Study.

| | | | | |
|---|---|---|---|---|
| Animal No. | Control | 20 mg/kg | 40 mg/kg | 75 mg/kg |

NOSE

| | | | | | | | | | | | | |
|---|---|---|---|---|---|---|---|---|---|---|---|---|
| Inflammation, diffuse | 2 | 2 | 3 | 1 | 2 | — | 3 | 3 | 2 | 2 | 3 | 1 |
| Degeneration, epithelium, focal | 2 | 1 | 2 | 1 | 1 | 1 | 2 | 3 | 2 | 1 | 2 | 1 |
| Necrosis, epithelium, multifocal | 1 | 1 | 2 | 2 | 1 | — | 3 | 3 | 2 | 2 | 3 | 1 |

LUNG

| | | | | | | | | | | | | |
|---|---|---|---|---|---|---|---|---|---|---|---|---|
| Infiltration, mononuclear cell, focal | — | — | 1 | — | — | — | — | 1 | 1 | 1 | 1 | — |
| Inflammation, granulomatous, focal | — | — | — | — | 2 | — | — | — | — | — | — | 1 |
| Infiltration, histocyte, multifocal | — | — | — | — | — | — | 2 | — | — | — | — | — |

TABLE 7
Blood Cell Analysis of a Single-dose Toxicity Study.

| | RBC | HGB | HCT | MCV | MCH | MCHC | RDW-SD | RDW-CV | RET |
|---|---|---|---|---|---|---|---|---|---|
| ID | (M/uL) | (g/dL) | (%) | (fL) | (pg) | (g/dL) | (fL) | (%) | (%) |
| #1 | 6.8 | 13.5 | 41.9 | 61.6 | 19.9 | 32.2 | 30.4 | 15.5 | 8.98 |
| #2 | 7.36 | 14.0 | 44.6 | 60.6 | 19.0 | 31.4 | 32.2 | 17.5 | 8.1 |
| #3 | 6.05 | 11.9 | 36.2 | 59.8 | 19.7 | 32.9 | 28.7 | 14.6 | 7.59 |
| #4 | 7.13 | 13.4 | 41.9 | 58.8 | 18.8 | 32.0 | 38.5 | 15.2 | 5.89 |
| #5 | 6.57 | 13.2 | 41.9 | 63.8 | 20.1 | 31.5 | 31.1 | 14.7 | 7.07 |
| #6 | 5.65 | 10.8 | 34.7 | 61.4 | 19.1 | 31.1 | 28.8 | 14.1 | 6.24 |
| #7 | 6.82 | 13.2 | 41.0 | 60.1 | 19.4 | 32.2 | 29.9 | 14.9 | 6.89 |
| #8 | 6.65 | 13.3 | 41.7 | 62.7 | 20.0 | 31.9 | 30.4 | 14.7 | 7.04 |
| #9 | 6.47 | 12.8 | 40.3 | 62.3 | 19.8 | 31.8 | 30.2 | 14.7 | 6.14 |
| #10 | 6.34 | 12.6 | 40.2 | 63.4 | 19.9 | 31.3 | 31.6 | 14.9 | 6.89 |
| #11 | 6.64 | 13.0 | 42.3 | 63.7 | 19.6 | 30.7 | 30.8 | 14.3 | 6.39 |
| #12 | 6.78 | 13.2 | 42.1 | 62.1 | 19.5 | 31.4 | 30.7 | 15.3 | 6.05 |

| | PLT | PDW | WBC | NEUT | LYMPH | MONO | EO | BASO |
|---|---|---|---|---|---|---|---|---|
| ID | (K/uL) | (fL) | (k/uL) | (%) | (%) | (%) | (%) | (%) |
| #1 | 1033 | 8.7 | 3.96 | 10.5 | 88.1 | 0.8 | 0.3 | 0.3 |
| #2 | 1064 | 8.9 | 4.52 | 26.1 | 70.6 | 2.7 | 0.4 | 0.2 |
| #3 | 1061 | 8.0 | 7.08 | 18.0 | 79.0 | 2.3 | 0.7 | 0.0 |
| #4 | 1064 | 8.2 | 2.56 | 14.8 | 80.5 | 2.7 | 1.2 | 0.8 |
| #5 | 1021 | 9.3 | 6.83 | 12.6 | 82.3 | 3.2 | 1.6 | 0.3 |
| #6 | 1055 | 8.7 | 3.76 | 13.3 | 82.7 | 2.9 | 1.1 | 0.0 |
| #7 | 1086 | 9.0 | 5.44 | 22.2 | 73.2 | 3.7 | 0.9 | 0.0 |
| #8 | 955 | 8.3 | 5.26 | 21.9 | 74.3 | 2.5 | 1.3 | 0.0 |
| #9 | 1052 | 8.4 | 5.01 | 15.2 | 81.6 | 2.0 | 1.0 | 0.2 |
| #10 | 1028 | 8.6 | 6.65 | 14.5 | 84.1 | 0.8 | 0.6 | 0.0 |
| #11 | 1142 | 8.8 | 6.13 | 14.6 | 83.0 | 2.1 | 0.3 | 0.0 |
| #12 | 1062 | 8.6 | 6.37 | 10.6 | 87.6 | 1.3 | 0.5 | 0.0 |

TABLE 8
Serum Analysis of a Single-dose Toxicity Study.

| | | AST | ALT | BUN | CREA |
|---|---|---|---|---|---|
| Treatment | ID | (U/L) | (U/L) | (mg/dL) | (mg/dL) |
| Control | A2 | 45 | 36 | 12.9 | 0.23 |
| | A3 | 57 | 58 | 15.6 | 0.33 |
| | A11 | 57 | 40 | 11.6 | 0.26 |
| 2 mg/kg | B1 | 62 | 40 | 15.1 | 0.27 |
| | B9 | 49 | 46 | 15.1 | 0.29 |
| | B12 | 47 | 47 | 19.2 | 0.26 |
| 10 mg/kg | C4 | 55 | 51 | 14.5 | 0.24 |
| | C8 | 52 | 31 | 11.6 | 0.19 |
| | C14 | 46 | 39 | 11.1 | 0.24 |
| 50 mg/kg | D16 | 46 | 34 | 13.5 | 0.23 |
| | D21 | 51 | 47 | 13.8 | 0.3 |
| | D25 | 57 | 40 | 13.1 | 0.24 |

Table 6 shows the histopathology study in rats (n=3) treated with single dose of C6G25S by intranasal instillation and the blood cells were collected at day 7 after treatment. Pathological change in nose and lung were evaluated. The severity grading scheme: 1=minimal (<10%) 2=mild (10-39%) 3=moderate (40-79%) 4=marked (80-100%). No abnormal findings are labeled as “-”.

Table 7 shows the hematology study in rats (n=3) treated with single dose of C6G25S by intranasal instillation and the blood cells were collected at day 7 after treatment. Control group (buffer alone) were labeled as 1-3, 20 mg/kg-treated group as 4-6, 40 mg/kg-treated group as 7-9, and 75 mg/kg-treated group as 10-12. RBC, RBC counts with millions per microliter; HGB, hemoglobin; HCT, hematocrit; MCV, mean corpuscular volume; MCHC, mean corpuscular hemoglobin concentration; RDW-SD, RBC distribution width standard deviation; RDW-CV, RBC distribution width with coefficient of variation; RET, reticulocyte equivalent; PLT, platelet count; PDW, platelet distribution width; WBC, white blood cells; NEUT, neutrophils; LYMPH, lymphocyte; MONO, monocyte; EO, eosinophils; BASO, basophils.

Table 8 shows the analysis of serum in rats (n=3) treated with single dose of C6G25S by intranasal instillation and serums were collected at day 7 after treatment. Control group (buffer alone) were labeled as 1-3, 20 mg/kg-treated group as 4-6, 40 mg/kg-treated group as 7-9, and 75 mg/kg-treated group as 10-12. AST, aspartate aminotransferase; ALT, alanine aminotransferase; BUN, blood urea nitrogen; CREA, creatinine.

The histopathology, hematology and blood biochemical analysis revealed no abnormalities in 14-day repeated-dose toxicity study. The results are provided in Tables 9-11 below. See also FIG. 11B.

TABLE 9
Histopathology of Major Tissue in Multiple-dose Toxicology Study.

| | | | | 2 mg/kg | | | 10 mg/kg | | | 50 mg/kg | | |
|---|---|---|---|---|---|---|---|---|---|---|---|---|
| | Control | | | C6G25S | | | C6G25S | | | C6G25S | | |
| Animal No. | A-2 | A-3 | A-11 | B-1 | B-9 | B-12 | C-4 | C-8 | C-14 | D-16 | D-21 | D-25 |
| Heart | — | — | — | — | — | — | — | — | — | — | — | — |

Liver

| | | | | | | | | | | | | |
|---|---|---|---|---|---|---|---|---|---|---|---|---|
| Infiltration, mononuclear cell, focal | — | 1 | — | — | 1 | — | — | — | — | 1 | — | 1 |
| Necrosis, focal | — | — | — | — | — | — | — | — | — | 1 | — | — |
| Spleen | — | — | | — | — | — | — | — | — | — | — | — |

Kidneys

| | | | | | | | |
|---|---|---|---|---|---|---|---|
| Infiltration, mononuclear cell, focal | 1 | | 2 | 1 | 2 | | |
| Basophilia, tubule, cortex, focal | | 1 | 1 | 1 | | 1 | 1 |
| Cyst, cortex, focal | | | | | | | |

Lung

| | | | | |
|---|---|---|---|---|
| Infiltration, mononuclear cell, focal | 1 | | 1 | |
| Inflammation, granulomatous, focal | 2 | 1 | | |
| Infiltration, histocyte, multifocal | | | | 1 |

Nasal cavity

| | | | | | | | | | | | |
|---|---|---|---|---|---|---|---|---|---|---|---|
| Inflammation, focal | | 2 | | 1 | | 1 | | 1 | | 1 | 1 |
| Degeneration, epithelium, focal | 2 | 2 | 3 | | 2 | 2 | 1 | 1 | 1 | 2 | — |

TABLE 10
Blood Cell Analysis of a Multiple-dose Toxicology Study.

| | RBC | HGB | HCT | MCV | MCH | MCHC | RDW-SD | RDW-CV | RET |
|---|---|---|---|---|---|---|---|---|---|
| ID | (M/uL) | (g/dL) | (%) | (fL) | (pg) | (g/dL) | (fL) | (%) | (%) |
| A2 | 8.51 | 13.0 | 42.6 | 50.1 | 15.3 | 30.5 | 27.9 | 21.3 | 3.28 |
| A3 | 8.38 | 13.0 | 44.5 | 53.1 | 15.5 | 29.2 | 27.9 | 20.3 | 3.51 |
| A11 | 7.99 | 12.2 | 41.4 | 51.8 | 15.3 | 29.5 | 26.1 | 19.6 | 3.36 |
| B12 | 8.30 | 13.2 | 42.8 | 51.6 | 15.9 | 30.8 | 26.6 | 20.5 | 3.57 |
| B9 | 7.67 | 11.6 | 38.7 | 50.5 | 15.1 | 30.0 | 26.2 | 18.4 | 4.02 |
| B12 | 8.26 | 13.8 | 45.0 | 54.5 | 16.7 | 30.7 | 28.4 | 19.9 | 4.08 |
| C4 | 9.10 | 14.2 | 47.3 | 52.0 | 15.6 | 30.0 | 26.2 | 21.1 | 3.58 |
| C8 | 7.72 | 11.9 | 39.9 | 51.7 | 15.4 | 29.8 | 26.2 | 18.8 | 3.16 |
| C14 | 8.34 | 13.3 | 44.0 | 52.8 | 15.9 | 30.2 | 27.3 | 20.7 | 2.82 |
| D16 | 7.49 | 12.0 | 39.1 | 52.2 | 16.0 | 30.7 | 25.3 | 17.2 | 2.42 |
| D21 | 8.15 | 12.5 | 41.9 | 51.4 | 15.3 | 29.8 | 27.2 | 20.9 | 0.62 |
| D25 | 8.17 | 12.5 | 41.3 | 50.6 | 15.3 | 30.3 | 25.5 | 18.7 | 3.55 |

| | PLT | PDW | WBC | NEUT | LYMPH | MONO | EO | BASO |
|---|---|---|---|---|---|---|---|---|
| ID | (K/uL) | (fL) | (k/uL) | (%) | (%) | (%) | (%) | (%) |
| A2 | 305 | 7.1 | 7.37 | 16.9 | 79.2 | 1.6 | 2.2 | 0.1 |
| A3 | 735 | 7.7 | 6.49 | 9.3 | 87.5 | 1.2 | 1.8 | 0.2 |
| A11 | 813 | 7.6 | 4.57 | 22.5 | 75.3 | 0.9 | 1.3 | 0.0 |
| B12 | 1254 | 6.5 | 6.03 | 13.6 | 83.7 | 1.3 | 1.2 | 0.2 |
| B9 | 1222 | 7.0 | 6.54 | 13.0 | 82.7 | 2.1 | 2.0 | 0.2 |
| B12 | 1160 | 7.2 | 5.17 | 12.0 | 82.8 | 1.9 | 3.3 | 0.0 |
| C4 | 778 | 6.9 | 5.14 | 11.7 | 84.2 | 1.4 | 2.3 | 0.4 |
| C8 | 943 | 6.9 | 5.81 | 16.8 | 79.9 | 1.0 | 2.1 | 0.2 |
| C14 | 51 | 9.5 | 3.27 | 18.7 | 75.2 | 3.7 | 2.1 | 0.3 |
| D16 | 498 | 6.5 | 4.41 | 7.9 | 89.6 | 0.7 | 1.6 | 0.2 |
| D21 | 77 | 7.8 | 6.52 | 14.1 | 78.8 | 4.0 | 2.9 | 0.2 |
| D25 | 978 | 6.9 | 5.30 | 14.0 | 81.1 | 3.2 | 1.5 | 0.2 |

TABLE 11
Serum Analysis of a Multiple-dose Toxicology Study.

| | | AST | ALT | BUN | CREA |
|---|---|---|---|---|---|
| Treatment | ID | (U/L) | (U/L) | (mg/dL) | (mg/dL) |
| Control | A2 | 168 | 50 | 23.1 | 0.33 |
| | A3 | 244 | 55 | 23.4 | 0.23 |
| | A11 | 75 | 27 | 22.2 | <0.20 |
| 2 mg/kg | B1 | 92 | 31 | 25.1 | 0.25 |
| | B9 | 83 | 34 | 30.4 | 0.22 |
| | B12 | 52 | 31 | 22.9 | <0.20 |
| 10 mg/kg | C4 | 38 | 26 | 25.3 | <0.20 |
| | C8 | 118 | 35 | 19.4 | 0.22 |
| | C14 | 69 | 28 | 29.7 | <0.20 |
| 50 mg/kg | D16 | 50 | 29 | 21.7 | 0.22 |
| | D21 | 220 | 38 | 21.3 | <0.20 |
| | D25 | 43 | 24 | 21.6 | <0.20 |

Table 9 shows the histopathology study in mice (n=3) that were intranasally administered with 2, 10 or 50 mg/kg of C6G25S once daily for 14 days and scarified for histopathology study. The control group were labeled as A2, A3, A11, 2 mg/kg-treated group as B1, B9, B12, 10 mg/kg-treated group as C4, C8, C14, and 50 mg/kg-treated group as D16, D21, D25. The severity grading scheme: 1=minimal (<10%), 2=mild (10-39%), 3=moderate (40-79%), 4=marked (80-100%). No abnormal findings are labeled as “-”.

Table 10 shows the blood cell analysis study in mice (n=3) that were intranasally administrated with 2, 10 or 50 mg/kg of C6G25S once daily for 14 days and blood were collected for blood cell analysis. Control group were labeled as A2, A3, A11, 2 mg/kg-treated group as B1, B9, B12, 10 mg/kg-treated group as C4, C8, C14, and 50 mg/kg-treated group as D16, D21, D25. RBC, RBC counts with millions per microliter; HGB, hemoglobin; HCT, hematocrit; MCV, mean corpuscular volume; MCHC, mean corpuscular hemoglobin concentration; RDW-SD, RBC distribution width standard deviation; RDW-CV, RBC distribution width with coefficient of variation; RET, reticulocyte equivalent; PLT, platelet count; PDW, platelet distribution width; WBC, white blood cells; NEUT, neutrophils; LYMPH, lymphocyte; MONO, monocyte; EO, eosinophils; BASO, basophils.

Table 11 shows the serum analysis of mice (n=3) that were intranasally administrated with 2, 10 or 50 mg/kg of C6G25S once daily for 14 days and serum were collected for analysis. The control group were labeled as A2, A3, A11, 2 mg/kg-treated group as B1, B9, B12, 10 mg/kg-treated group as C4, C8, C14, and 50 mg/kg-treated group as D16, D21, D25. AST, aspartate aminotransferase; ALT, alanine aminotransferase; BUN, blood urea nitrogen; CREA, creatinine. Index: H=Hemolysis; L=Lipemia; F=Fibrin, N/A=Not observed abnormal. CREA: The linear range is 0.2˜25 mg/dL, less than the linear range is presented as <0.20 mg/dL.

### VIII. Therapeutic Effects of C6G25S.

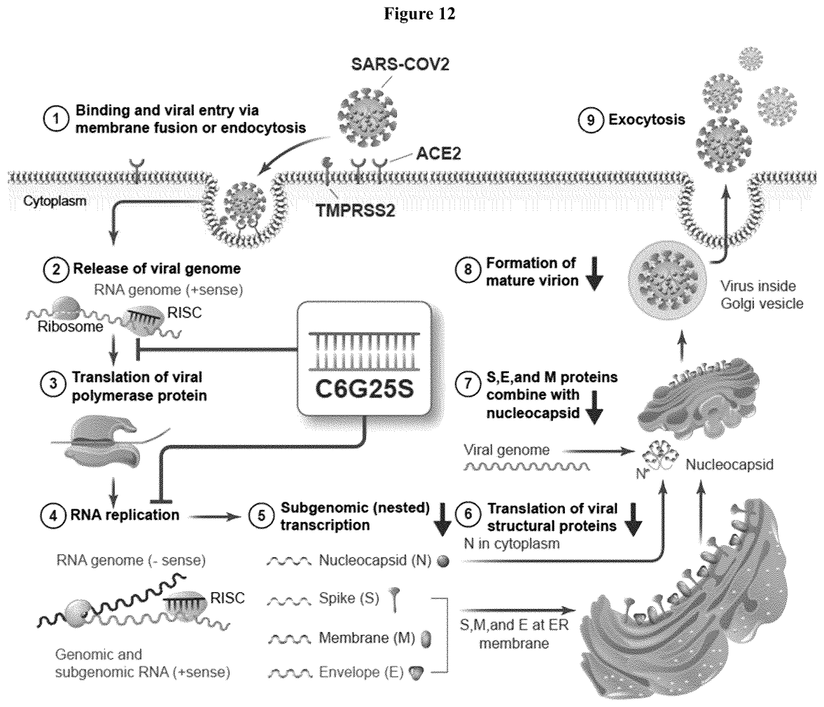

C6G25S targets a highly conserved RdRp region of SARS-CoV-1/2 as shown in FIG. 4A. The potential mechanism of action of C6G25S in inhibiting SARS-CoV-1/2 infection is illustrated in FIG. 12. SARS-CoV-2 binds to ACE2 receptors on the host cell and induces endocytosis. Cleavage of the viral spike protein by TMPRSS2 triggers membrane fusion and the viral sense (+) RNA genome is released, hijacking the host's ribosome to produce RNA-dependent RNA polymerase and replicate. Meanwhile, subgenomic transcription and translation generate large amounts of viral structural proteins, such as the nucleocapsid, spike, membrane, and envelope. The progeny virus assembles, and mature virions are released by exocytosis. C6G25S can interact with the RNA-induced silencing complex to digest the viral genome's RNA and polymerase mRNA through the RNAi effect. By reducing the copy number of the viral genome and polymerase mRNA, the subsequent steps involved in the virus's replication cycle are indirectly inhibited, thereby halting SARS-CoV-2 infection.

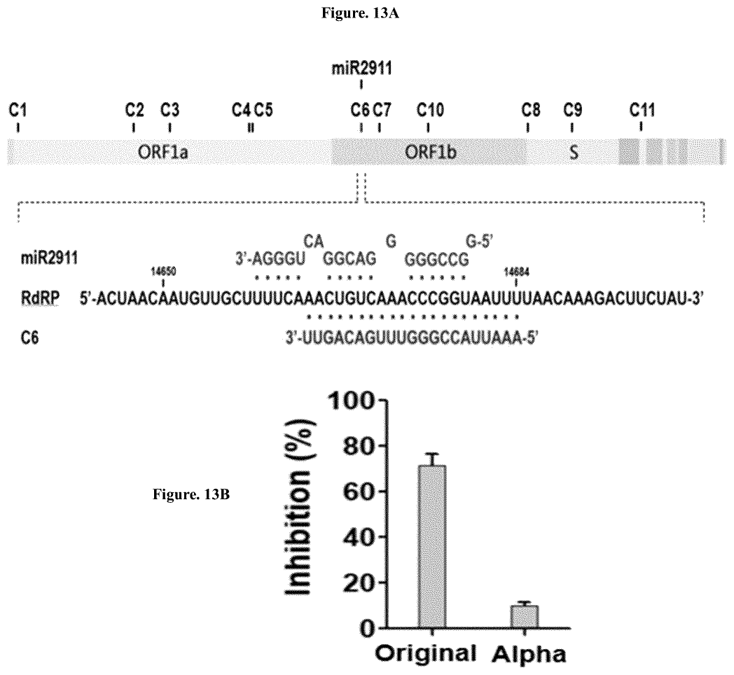

Interestingly, it was also found that miR-2911, a natural microRNA that had been reported to inhibit SARS-CoV-2 (Zhou, et al., 2020, Cell Discov 6: 54), had a predicted target site overlapping with C6 (FIG. 13A), but it was shown to only reduce 72% of original virus and was unable to inhibit alpha variant in in vitro assay (FIG. 13B). This finding suggests that C6G25S is a much promising therapeutic compared to miR-2911.

The coverage rate of C6G25S with respect to SAR-CoV-2 variants was found to be 99.8% when using over 200,000 SARS-CoV-2 genome sequences downloaded from the National Center for Biotechnology Information on Aug. 22, 2021. 200,000 genome sequences of SARS-CoV2 were downloaded from NCBI virus SARS-CoV-2 Data Hub at Aug. 22, 2021. The results for the are provided in Table 12 below.

TABLE 12
Coverage Rates of C6G25S Against SARS-CoV-2 Variants

| SARS-CoV-2 Variants | Percentage | Coverage Rates |
|---|---|---|
| Alpha* | 78.83% | 99.9% |
| Delta | 6.43% | 99.8% |
| Epsilon | 4.88% | 99.9% |
| Gamma | 3.31% | 99.9% |
| Lota | 5.61% | 99.9% |
| Others | 0.94% | 99.5% |

*The C to U transversion of Alpha variants binding to the 9^{th} nucleotide of antisense C6G25S was tolerated for the calculation of coverage rate.

In sum, the results provided in this example demonstrate that C6G25S, as an exemplary siRNA targeting SARS-CoV-2 (e.g., targeting a highly conserved RdRp region of the virus) could effectively inhibit infection of various SARS-CoV-2 strains via RNA interference to cleave the complementary viral RNA at the recognition site. The results show that C6G25S can tackle multiple SARS-CoV-2 variants (see Table 12 above) with a picomolar range of IC₅₀. The inhibitory activity of C6G25S is more potent than a naturally-occurring miRNA miR-2911, which has a predicted targeting site overlapping with the target site of C6G25S.

Delivery of the siRNA via, for example, aerosol inhalation (AI) showed uniform distribution of the siRNA across the entire lung, while intranasal instillation (IN) showed high dosing efficiency within the nasal cavity, suggesting that a combined delivery route would be expected to be more effective for prophylactic and/or actual treatment of SARS-CoV-2 infection. An averaged 99.9% reduction of viral RNA and no measurable plaque-forming virions were detected after the prophylactic treatment. In the post-exposure treatment, viral RNA was reduced by 96.2% and infectious virions by 96.1% via inhalation. Moreover, spike protein expression and immune cell infiltration in the lungs of infected mice receiving C6G25S treatment were both significantly decreased, along with reductions of disease-associated pathological features. These results all suggest that the anti-SARS-CoV-2 siRNAs disclosed herein, including C6G25S as an example, would be effective in both prophylactic treatment and in treating patients who have been infected with the virus.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for inhibiting a severe acute respiratory syndrome coronavirus (SARS-CoV), the method comprising:
contacting an effective amount of a small interfering RNA (siRNA) with a cell infected with a SARS-CoV virus,
wherein the siRNA targets a genomic site of a SARS-CoV virus, which optionally is SARS-CoV-1 or SARS-CoV-2.

2. The method of claim 1, wherein the genomic site is in a SARS-CoV gene selected from the group consisting of POL gene, Spike gene, Helicase gene, and Envelop gene.

3. The method of claim 1, wherein the genomic site comprises the nucleotide sequence selected from the group consisting of:
(i)
(SEQ ID NO: 2)
5′-GAGGCACGUCAACAUCUUA-3′,
(ii)
(SEQ ID NO: 4)
5′-CAGCAUUAAAUCACACUAA-3′,
(iii)
(SEQ ID NO: 6)
5′-CGGUGUUUAAACCGUGUUU-3′,
(iv)
(SEQ ID NO: 8)
5′-GUGGUACAACUACACUUAA-3′,
(v)
(SEQ ID NO: 10)
5′-UGGCUUGAUGACGUAGUUU-3′,
(vi)
(SEQ ID NO: 12)
5′-CUGUCAAACCCGGUAAUUU-3′,
(vii)
(SEQ ID NO: 14)
5′-GCGGUUCACUAUAUGUUAA-3′,
(viii)
(SEQ ID NO: 16)
5′-GCCACUAGUCUCUAGUCAG-3′,
(ix)
(SEQ ID NO: 18)
5′-CUCCUACUUGGCGUGUUUA-3′,
(x)
(SEQ ID NO: 20)
5′-CGCACAUUGCUAACUAAGG-3′,
and
(xi)
(SEQ ID NO: 22)
5′-CAGGUACGUUAAUAGUUAA-3′

4. The method of claim 1, wherein the siRNA targets a site in an RNA-dependent RNA polymerase (RdRP) gene of the SARS-CoV virus.

5. The method of claim 4, wherein the siRNA targets a site in the RdRP messenger RNA (mRNA).

6. The method of claim 5, wherein the siRNA targets a site in the mRNA, and wherein the target site is in the nucleotide sequence of 5′-UUGCUUUUCAAACUGUCAAACCCGGUAAUUUUAACAAAGA-3′ (SEQ ID NO: 23).

7. The method of claim 6, wherein the target site is in the nucleotide sequence of 5′-UUUCAAACUGUCAAACCCGGUAAUUUU-3′ (SEQ ID NO: 24).

8. The method of claim 1, wherein the siRNA is a double-strand molecule comprising a sense chain and an antisense chain.

9. The method of claim 8, wherein the sense chain and the antisense chain comprises the following nucleotide sequences, respectively:
(i)
(SEQ ID NO: 25)
5′-GAGGCACGUCAACAUCUUX₁-3′
and
(SEQ ID NO: 26)
5′-X₂AAGAUGUUGACGUGCCUCN₁N₂-3′;
(ii)
(SEQ ID NO: 27)
5′-CAGCAUUAAAUCACACUAX₁-3′,
and
(SEQ ID NO: 28)
5′-X₂UAGUGUGAUUUAAUGCUGN₁N₂-3′;
(iii)
(SEQ ID NO: 29)
5′-CGGUGUUUAAACCGUGUUX₁-3′,
and
(SEQ ID NO: 30)
5′-X₂AACACGGUUUAAACACCGN₁N₂-3′;
(iv)
(SEQ ID NO: 31)
5′-GUGGUACAACUACACUUAX₁-3′,
and
(SEQ ID NO: 32)
5′-X₂UAAGUGUAGUUGUACCACN₁N₂-3′,
(v)
(SEQ ID NO: 33)
5′-UGGCUUGAUGACGUAGUUX₁-3′,
and
(SEQ ID NO: 34)
5′-X₂AACUACGUCAUCAAGCCAN₁N₂-3′;
(vi)
(SEQ ID NO: 35)
5′-CUGUCAAACCCGGUAAUUX₁-3′,
and
(SEQ ID NO: 36)
5′-X₂AAUUACCGGGUUUGACAGN₁N₂-3′;
(vii)
(SEQ ID NO: 37)
5′-GCGGUUCACUAUAUGUUAX₁-3′,
and
(SEQ ID NO: 38)
5′-X₂UAACAUAUAGUGAACCGCN₁N₂-3′;
(viii)
(SEQ ID NO: 39)
5′-GCCACUAGUCUCUAGUCAX₁-3′,
and
(SEQ ID NO: 40)
5′-X₂UGACUAGAGACUAGUGGCN₁N₂-3′;
(ix)
(SEQ ID NO: 41)
5′-CUCCUACUUGGCGUGUUUX₁-3′,
and
(SEQ ID NO: 42)
5′-X₂AAACACGCCAAGUAGGAGN₁N₂-3′;
(x)
(SEQ ID NO: 43)
5′-CGCACAUUGCUAACUAAGX₁-3′,
and
(SEQ ID NO: 44)
5′-X₂CUUAGUUAGCAAUGUGCGN₁N₂-3′;
or
(xi)
(SEQ ID NO: 45)
5′-CAGGUACGUUAAUAGUUAX₁-3′
and
(SEQ ID NO: 46)
5′-X₂UAACUAUUAACGUACCUGN₁N₂-3′;
wherein X₁ and X₂ in each of the sense chain and antisense chain of each of (i)-(xi), independently, are A and U, respectively or vice versa, or G and C, respectively, or vice versa; and
wherein each of N₁ and N₂ in each of the sense chain and antisense chain of each of (i)-(xi), independently, is A, U, G, or C; optionally wherein N₂ is U.

10. The method of claim 6, wherein the sense chain and the antisense chain comprises the following nucleotide sequences, respectively:
(i)
(SEQ ID NO: 25)
5′-GAGGCACGUCAACAUCUUX₁-3′
and
(SEQ ID NO: 47)
5′-X₂AAGAUGUUGACGUGCCUCUU-3′;
(ii)
(SEQ ID NO: 27)
5′-CAGCAUUAAAUCACACUAX₁-3′,
and
(SEQ ID NO: 48)
5′-X₂UAGUGUGAUUUAAUGCUGUU-3′;
(iii)
(SEQ ID NO: 29)
5′-CGGUGUUUAAACCGUGUUX₁-3′,
and
(SEQ ID NO: 49)
5′-X₂AACACGGUUUAAACACCGUU-3′;
(iv)
(SEQ ID NO: 31)
5′-GUGGUACAACUACACUUAX₁-3′,
and
(SEQ ID NO: 50)
5′-X₂UAAGUGUAGUUGUACCACUU-3′;
(v)
(SEQ ID NO: 33)
5′-UGGCUUGAUGACGUAGUUX₁-3′,
and
(SEQ ID NO: 51)
5′-X₂AACUACGUCAUCAAGCCAUU-3′;
(vi)
(SEQ ID NO: 35)
5′-CUGUCAAACCCGGUAAUUX₁-3′,
and
(SEQ ID NO: 52)
5′-X₂AAUUACCGGGUUUGACAGUU-3′;
(vii)
(SEQ ID NO: 37)
5′-GCGGUUCACUAUAUGUUAX₁-3′,
and
(SEQ ID NO: 53)
5′-X₂UAACAUAUAGUGAACCGCUU-3′;
(viii)
(SEQ ID NO: 39)
5′-GCCACUAGUCUCUAGUCAX₁-3′,
and
(SEQ ID NO: 54)
5′-X₂UGACUAGAGACUAGUGGCUU-3′;
(ix)
(SEQ ID NO: 41)
5′-CUCCUACUUGGCGUGUUUX₁-3′,
and
(SEQ ID NO: 55)
5′-X₂AAACACGCCAAGUAGGAGUU-3′;
(x)
(SEQ ID NO: 43)
5′-CGCACAUUGCUAACUAAGX₁-3′,
and
(SEQ ID NO: 56)
5′-X₂CUUAGUUAGCAAUGUGCGUU-3′;
or
(xi)
(SEQ ID NO: 45)
5′-CAGGUACGUUAAUAGUUAX₁-3′
and
(SEQ ID NO: 57)
5′-X₂UAACUAUUAACGUACCUGUU-3′;
wherein X₁ and X₂ in each of the sense chain and antisense chain of each of (i)-(xi), independently, are A and U, respectively, or vice versa.

11. The method of claim 9, wherein the sense chain and the antisense chain comprise the nucleotide sequences set forth in (vi), (vii), (viii), (x), or (xi).

12. The method of claim 1, wherein the siRNA is selected from the group consisting of C6, C7, C8, C10, and C11, optionally wherein the siRNA is C6.

13. The method of claim 1, wherein the siRNA comprises one or more modified nucleotides.

14. The method of claim 13, wherein the one or more modified nucleotides comprise 2′-fluoro, 2′-O-methyl, or a combination thereof.

15. The method of claim 1, wherein the siRNA comprises one or more phosphorothioate bonds.

16. The method of claim 13, wherein the siRNA is C6G25S, C8G25S, or C10G31A, optionally wherein the siRNA is C6G25S.

17. The method of claim 1, wherein the contacting step is performed by administering the siRNA to a subject having infected by the SARS-CoV virus.

18. The method of claim 17, wherein the siRNA is formulated in a pharmaceutical composition, which further comprises a pharmaceutically acceptable carrier.

19. The method of claim 17, wherein the subject is a human patient having COVID-19.

20. The method of claim 17, wherein the subject is further administered an agent for treatment of infection caused by the SARS-CoV, which optionally is SARS-CoV-1 or SARS-CoV-2, preferably wherein the infection is caused by SARS-CoV-2.

21. The method of claim 20, wherein the agent comprises an anti-SARS-CoV-2 antibody, remdesivir, a steroid, an anti-SARS-CoV vaccine, or a combination thereof.

22. The method of claim 17, wherein the subject is a human patient at risk for SARS-CoV infection.

23. The method of claim 17, wherein the siRNA is administered to the subject by intranasal instillation, aerosol inhalation, or a combination thereof.

24. A small interfering RNA (siRNA) that targets a SARS-CoV virus, wherein the siRNA comprises a sequence complementary to a genomic site of the SARS-CoV virus, wherein the siRNA is set forth in claim 1.

25. A pharmaceutical composition comprising a small interfering RNA of claim 22 and a pharmaceutically acceptable carrier.


SEQUENCE LISTING (75 sequences)

SEQ ID NO: 1 - RNA, 21 nt, Artificial Sequence
  Synthetic
uaagauguugacgugccucuu

SEQ ID NO: 2 - RNA, 19 nt, Artificial Sequence
  Synthetic
gaggcacgucaacaucuua

SEQ ID NO: 3 - RNA, 21 nt, Artificial Sequence
  Synthetic
uuagugugauuuaaugcuguu

SEQ ID NO: 4 - RNA, 19 nt, Artificial Sequence
  Synthetic
cagcauuaaaucacacuaa

SEQ ID NO: 5 - RNA, 21 nt, Artificial Sequence
  Synthetic
aaacacgguuuaaacaccguu

SEQ ID NO: 6 - RNA, 19 nt, Artificial Sequence
  Synthetic
cgguguuuaaaccguguuu

SEQ ID NO: 7 - RNA, 21 nt, Artificial Sequence
  Synthetic
uuaaguguaguuguaccacuu

SEQ ID NO: 8 - RNA, 19 nt, Artificial Sequence
  Synthetic
gugguacaacuacacuuaa

SEQ ID NO: 9 - RNA, 21 nt, Artificial Sequence
  Synthetic
aaacuacgucaucaagccauu

SEQ ID NO: 10 - RNA, 19 nt, Artificial Sequence
  Synthetic
uggcuugaugacguaguuu

SEQ ID NO: 11 - RNA, 21 nt, Artificial Sequence
  Synthetic
aaauuaccggguuugacaguu

SEQ ID NO: 12 - RNA, 19 nt, Artificial Sequence
  Synthetic
cugucaaacccgguaauuu

SEQ ID NO: 13 - RNA, 21 nt, Artificial Sequence
  Synthetic
uuaacauauagugaaccgcuu

SEQ ID NO: 14 - RNA, 19 nt, Artificial Sequence
  Synthetic
gcgguucacuauauguuaa

SEQ ID NO: 15 - RNA, 21 nt, Artificial Sequence
  Synthetic
uugacuagagacuaguggcuu

SEQ ID NO: 16 - RNA, 19 nt, Artificial Sequence
  Synthetic
gccacuagucucuagucag

SEQ ID NO: 17 - RNA, 21 nt, Artificial Sequence
  Synthetic
uaaacacgccaaguaggaguu

SEQ ID NO: 18 - RNA, 19 nt, Artificial Sequence
  Synthetic
cuccuacuuggcguguuua

SEQ ID NO: 19 - RNA, 21 nt, Artificial Sequence
  Synthetic
ucuuaguuagcaaugugcguu

SEQ ID NO: 20 - RNA, 19 nt, Artificial Sequence
  Synthetic
cgcacauugcuaacuaagg

SEQ ID NO: 21 - RNA, 21 nt, Artificial Sequence
  Synthetic
uuaacuauuaacguaccuguu

SEQ ID NO: 22 - RNA, 19 nt, Artificial Sequence
  Synthetic
cagguacguuaauaguuaa

SEQ ID NO: 23 - RNA, 40 nt, Artificial Sequence
  Synthetic
uugcuuuucaaacugucaaacccgguaauuuuaacaaaga

SEQ ID NO: 24 - RNA, 27 nt, Artificial Sequence
  Synthetic
uuucaaacugucaaacccgguaauuuu

SEQ ID NO: 25 - RNA, 19 nt, Artificial Sequence
  Synthetic
  misc_feature (19)..(19): n is a, c, g, or u
gaggcacgucaacaucuun

SEQ ID NO: 26 - RNA, 21 nt, Artificial Sequence
  Synthetic
  misc_feature (1)..(1): n is a, c, g, or u
  misc_feature (20)..(21): n is a, c, g, or u
naagauguugacgugccucnn

SEQ ID NO: 27 - RNA, 19 nt, Artificial Sequence
  Synthetic
  misc_feature (19)..(19): n is a, c, g, or u
cagcauuaaaucacacuan

SEQ ID NO: 28 - RNA, 21 nt, Artificial Sequence
  Synthetic
  misc_feature (1)..(1): n is a, c, g, or u
  misc_feature (20)..(21): n is a, c, g, or u
nuagugugauuuaaugcugnn

SEQ ID NO: 29 - RNA, 19 nt, Artificial Sequence
  Synthetic
  misc_feature (19)..(19): n is a, c, g, or u
cgguguuuaaaccguguun

SEQ ID NO: 30 - RNA, 21 nt, Artificial Sequence
  Synthetic
  misc_feature (1)..(1): n is a, c, g, or u
  misc_feature (20)..(21): n is a, c, g, or u
naacacgguuuaaacaccgnn

SEQ ID NO: 31 - RNA, 19 nt, Artificial Sequence
  Synthetic
  misc_feature (19)..(19): n is a, c, g, or u
gugguacaacuacacuuan

SEQ ID NO: 32 - RNA, 21 nt, Artificial Sequence
  Synthetic
  misc_feature (1)..(1): n is a, c, g, or u
  misc_feature (20)..(21): n is a, c, g, or u
nuaaguguaguuguaccacnn

SEQ ID NO: 33 - RNA, 19 nt, Artificial Sequence
  Synthetic
  misc_feature (19)..(19): n is a, c, g, or u
uggcuugaugacguaguun

SEQ ID NO: 34 - RNA, 21 nt, Artificial Sequence
  Synthetic
  misc_feature (1)..(1): n is a, c, g, or u
  misc_feature (20)..(21): n is a, c, g, or u
naacuacgucaucaagccann

SEQ ID NO: 35 - RNA, 19 nt, Artificial Sequence
  Synthetic
  misc_feature (19)..(19): n is a, c, g, or u
cugucaaacccgguaauun

SEQ ID NO: 36 - RNA, 21 nt, Artificial Sequence
  Synthetic
  misc_feature (1)..(1): n is a, c, g, or u
  misc_feature (20)..(21): n is a, c, g, or u
naauuaccggguuugacagnn

SEQ ID NO: 37 - RNA, 19 nt, Artificial Sequence
  Synthetic
  misc_feature (19)..(19): n is a, c, g, or u
gcgguucacuauauguuan

SEQ ID NO: 38 - RNA, 21 nt, Artificial Sequence
  Synthetic
  misc_feature (1)..(1): n is a, c, g, or u
  misc_feature (20)..(21): n is a, c, g, or u
nuaacauauagugaaccgcnn

SEQ ID NO: 39 - RNA, 19 nt, Artificial Sequence
  Synthetic
  misc_feature (19)..(19): n is a, c, g, or u
gccacuagucucuagucan

SEQ ID NO: 40 - RNA, 21 nt, Artificial Sequence
  Synthetic
  misc_feature (1)..(1): n is a, c, g, or u
  misc_feature (20)..(21): n is a, c, g, or u
nugacuagagacuaguggcnn

SEQ ID NO: 41 - RNA, 19 nt, Artificial Sequence
  Synthetic
  misc_feature (19)..(19): n is a, c, g, or u
cuccuacuuggcguguuun

SEQ ID NO: 42 - RNA, 21 nt, Artificial Sequence
  Synthetic
  misc_feature (1)..(1): n is a, c, g, or u
  misc_feature (20)..(21): n is a, c, g, or u
naaacacgccaaguaggagnn

SEQ ID NO: 43 - RNA, 19 nt, Artificial Sequence
  Synthetic
  misc_feature (19)..(19): n is a, c, g, or u
cgcacauugcuaacuaagn

SEQ ID NO: 44 - RNA, 21 nt, Artificial Sequence
  Synthetic
  misc_feature (1)..(1): n is a, c, g, or u
  misc_feature (20)..(21): n is a, c, g, or u
ncuuaguuagcaaugugcgnn

SEQ ID NO: 45 - RNA, 19 nt, Artificial Sequence
  Synthetic
  misc_feature (19)..(19): n is a, c, g, or u
cagguacguuaauaguuan

SEQ ID NO: 46 - RNA, 21 nt, Artificial Sequence
  Synthetic
  misc_feature (1)..(1): n is a, c, g, or u
  misc_feature (20)..(21): n is a, c, g, or u
nuaacuauuaacguaccugnn

SEQ ID NO: 47 - RNA, 21 nt, Artificial Sequence
  Synthetic
  misc_feature (1)..(1): n is a, c, g, or u
naagauguugacgugccucuu

SEQ ID NO: 48 - RNA, 21 nt, Artificial Sequence
  Synthetic
  misc_feature (1)..(1): n is a, c, g, or u
nuagugugauuuaaugcuguu

SEQ ID NO: 49 - RNA, 21 nt, Artificial Sequence
  Synthetic
  misc_feature (1)..(1): n is a, c, g, or u
naacacgguuuaaacaccguu

SEQ ID NO: 50 - RNA, 21 nt, Artificial Sequence
  Synthetic
  misc_feature (1)..(1): n is a, c, g, or u
nuaaguguaguuguaccacuu

SEQ ID NO: 51 - RNA, 21 nt, Artificial Sequence
  Synthetic
  misc_feature (1)..(1): n is a, c, g, or u
naacuacgucaucaagccauu

SEQ ID NO: 52 - RNA, 21 nt, Artificial Sequence
  Synthetic
  misc_feature (1)..(1): n is a, c, g, or u
naauuaccggguuugacaguu

SEQ ID NO: 53 - RNA, 21 nt, Artificial Sequence
  Synthetic
  misc_feature (1)..(1): n is a, c, g, or u
nuaacauauagugaaccgcuu

SEQ ID NO: 54 - RNA, 21 nt, Artificial Sequence
  Synthetic
  misc_feature (1)..(1): n is a, c, g, or u
nugacuagagacuaguggcuu

SEQ ID NO: 55 - RNA, 21 nt, Artificial Sequence
  Synthetic
  misc_feature (1)..(1): n is a, c, g, or u
naaacacgccaaguaggaguu

SEQ ID NO: 56 - RNA, 21 nt, Artificial Sequence
  Synthetic
  misc_feature (1)..(1): n is a, c, g, or u
ncuuaguuagcaaugugcguu

SEQ ID NO: 57 - RNA, 21 nt, Artificial Sequence
  Synthetic
  misc_feature (1)..(1): n is a, c, g, or u
nuaacuauuaacguaccuguu

SEQ ID NO: 58 - RNA, 21 nt, Artificial Sequence
  Synthetic
uugacaguuugggccauuaaa

SEQ ID NO: 59 - RNA, 50 nt, Artificial Sequence
  Synthetic
auguugcuuuucaaacugucaaacccgguaauuuuaacaaagacuucuau

SEQ ID NO: 60 - RNA, 20 nt, Artificial Sequence
  Synthetic
agggucaggcagggggccgg

SEQ ID NO: 61 - RNA, 57 nt, Artificial Sequence
  Synthetic
acuaacaauguugcuuuucaaacugucaaacccgguaauuuuaacaaagacuucuau

SEQ ID NO: 62 - DNA, 26 bp, Artificial Sequence
  Synthetic
acaggtacgttaatagttaatagcgt

SEQ ID NO: 63 - DNA, 22 bp, Artificial Sequence
  Synthetic
acattgcagcagtacgcacaca

SEQ ID NO: 64 - DNA, 26 bp, Artificial Sequence
  Synthetic
acactagccatccttactgcgcttcg

SEQ ID NO: 65 - DNA, 52 bp, Artificial Sequence
  Synthetic
gtcgtatccagtgcagggtccgaggtattcgcactggatacgacaactgtca

SEQ ID NO: 66 - DNA, 21 bp, Artificial Sequence
  Synthetic
aagcgcctaaattaccgggtt

SEQ ID NO: 67 - DNA, 16 bp, Artificial Sequence
  Synthetic
gtgcagggtccgaggt

SEQ ID NO: 68 - RNA, 19 nt, Artificial Sequence
  Synthetic
  misc_feature (1)..(4): modified with 2′-O-methylation
  misc_feature (1)..(3): modified with phosphorothioate
  misc_feature (5)..(5): modified with 2′-fluoro
  misc_feature (6)..(6): modified with 2′-O-methylation
  misc_feature (7)..(10): modified with 2′-fluoro
  misc_feature (11)..(19): modified with 2′-O-methylation
cugucaaacccgguaauuu

SEQ ID NO: 69 - RNA, 21 nt, Artificial Sequence
  Synthetic
  misc_feature (1)..(1): modified with 2′-O-methylation
  misc_feature (1)..(3): modified with phosphorothioate
  misc_feature (2)..(2): modified with 2′-fluoro
  misc_feature (3)..(3): modified with 2′-O-methylation
  misc_feature (4)..(5): modified with 2′-fluoro
  misc_feature (6)..(7): modified with 2′-O-methylation
  misc_feature (8)..(9): modified with 2′-fluoro
  misc_feature (10)..(13): modified with 2′-O-methylation
  misc_feature (14)..(14): modified with 2′-fluoro
  misc_feature (15)..(15): modified with 2′-O-methylation
  misc_feature (16)..(16): modified with 2′-fluoro
  misc_feature (17)..(17): modified with 2′-O-methylation
  misc_feature (18)..(18): modified with 2′-fluoro
  misc_feature (19)..(21): modified with phosphorothioate
  misc_feature (19)..(21): modified with 2′-O-methylation
aaauuaccggguuugacaguu

SEQ ID NO: 70 - RNA, 19 nt, Artificial Sequence
  Synthetic
  misc_feature (1)..(4): modified with 2′-O-methylation
  misc_feature (1)..(3): modified with phosphorothioate
  misc_feature (5)..(5): modified with 2′-fluoro
  misc_feature (6)..(6): modified with 2′-O-methylation
  misc_feature (7)..(10): modified with 2′-fluoro
  misc_feature (11)..(19): modified with 2′-O-methylation
gccacuagucucuagucaa

SEQ ID NO: 71 - RNA, 21 nt, Artificial Sequence
  Synthetic
  misc_feature (1)..(1): modified with 2′-O-methylation
  misc_feature (1)..(3): modified with phosphorothioate
  misc_feature (2)..(2): modified with 2′-fluoro
  misc_feature (3)..(3): modified with 2′-O-methylation
  misc_feature (4)..(5): modified with 2′-fluoro
  misc_feature (6)..(7): modified with 2′-O-methylation
  misc_feature (8)..(9): modified with 2′-fluoro
  misc_feature (10)..(13): modified with 2′-O-methylation
  misc_feature (14)..(14): modified with 2′-fluoro
  misc_feature (15)..(15): modified with 2′-O-methylation
  misc_feature (16)..(16): modified with 2′-fluoro
  misc_feature (17)..(17): modified with 2′-O-methylation
  misc_feature (18)..(18): modified with 2′-fluoro
  misc_feature (19)..(21): modified with phosphorothioate
  misc_feature (19)..(21): modified with phosphorothioate
  misc_feature (19)..(21): modified with 2′-O-methylation
uugacuagagacuaguggcuu

SEQ ID NO: 72 - RNA, 19 nt, Artificial Sequence
  Synthetic
  misc_feature (4)..(4): modified with 2′-O-methylation
  misc_feature (4)..(6): modified with phosphorothioate
  misc_feature (5)..(5): modified with 2′-fluoro
  misc_feature (6)..(6): modified with 2′-O-methylation
  misc_feature (7)..(7): modified with 2′-fluoro
  misc_feature (8)..(8): modified with 2′-O-methylation
  misc_feature (9)..(9): modified with 2′-fluoro
  misc_feature (10)..(10): modified with 2′-O-methylation
  misc_feature (11)..(11): modified with 2′-fluoro
  misc_feature (12)..(19): modified with 2′-O-methylation
  misc_feature (17)..(19): modified with phosphorothioate
cgcacauugcuaacuaaga

SEQ ID NO: 73 - RNA, 21 nt, Artificial Sequence
  Synthetic
  misc_feature (1)..(1): modified with 2′-O-methylation
  misc_feature (1)..(3): modified with phosphorothioate
  misc_feature (2)..(2): modified with 2′-fluoro
  misc_feature (3)..(3): modified with 2′-O-methylation
  misc_feature (4)..(4): modified with 2′-fluoro
  misc_feature (5)..(5): modified with 2′-O-methylation
  misc_feature (6)..(6): modified with 2′-fluoro
  misc_feature (7)..(7): modified with 2′-O-methylation
  misc_feature (8)..(8): modified with 2′-fluoro
  misc_feature (10)..(10): modified with 2′-fluoro
  misc_feature (11)..(11): modified with 2′-O-methylation
  misc_feature (12)..(12): modified with 2′-fluoro
  misc_feature (13)..(13): modified with 2′-O-methylation
  misc_feature (14)..(14): modified with 2′-fluoro
  misc_feature (15)..(15): modified with 2′-O-methylation
  misc_feature (16)..(16): modified with 2′-fluoro
  misc_feature (17)..(21): modified with 2′-O-methylation
  misc_feature (19)..(21): modified with phosphorothioate
ucuuaguuagcaaugugcguu

SEQ ID NO: 74 - RNA, 19 nt, Artificial Sequence
  Synthetic
gccacuagucucuagucaa

SEQ ID NO: 75 - RNA, 19 nt, Artificial Sequence
  Synthetic
cgcacauugcuaacuaaga